(19) United States
(12) Patent Application Publication
(10) Pub. No.: US 2004/0265808 A1
(43) Pub. Date: Dec. 30, 2004

(54) Genes involved in osteogenesis, and methods of use

(76) Inventors: Teresa Garcia, Champigny Sur Marne (FR); Sergio Roman Roman, Paris (FR); Roland Baron, Guilforg (FR); Katherine Call, Malden, MA (US); Joachim Theilhaber, Cambridge, MA (US); Timothy Connolly, Westwood, MA (US); Amanda Jackson, Paris (FR); Steven E Bushnell, Medfield, MA (US); Georges Rawadi, Paris (FR)

Correspondence Address: Patricia D Granados, Heller Ehrman White & McAuliffe, Suite 300, 1666 K Street N W, Washington, DC, 20006, US

(21) Appl. No.: 10/473,974

(22) PCT Filed: Apr. 5, 2002
(86) PCT No.: PCT/IB02/02211

Related U.S. Application Data
(60) Provisional application No. 60/281,400, filed on Apr. 5, 2001.

(51) Int. Cl. C12Q 1/68

(52) U.S. Cl. 435/6

(57) ABSTRACT

The present invention relates to methods of diagnosis, therapy, and screening of new therapeutic compounds in the field of osteogenesis, based on the differential expression observed for the genes of the invention, represented by SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

### RELATED APPLICATIONS

This application relates to the U.S. Provisional Patent Application 60/281,400 filed on Apr. 5, 2001.

### FIELD OF THE INVENTION

The present invention relates to methods of diagnosis, therapy, and screening of new therapeutic compounds in the field of osteogenesis.

### BACKGROUND OF THE INVENTION

Osteoporosis is a chronic-degenerative and incapacitating condition of generalized skeletal fragility due to a reduction in the amount of bone and a disruption of skeletal microarchitecture to the point that fracture vulnerability increases. It is a frequent osteometabolic disease, with a high morbidity, frequently associated with hip and vertebral fractures.

Osteoporosis is actually a syndrome, since there are a lot of conditions that can lead to this state of bone fragility. It is a national health problem due to its high prevalence and incapacitating complications, such as pain and fractures. Its prevention can avoid the large expenditure caused by the treatment of the resulting pathological fractures

The increase in life expectation, specially in developed countries, is causing a similar increase in the prevalence of osteoporosis. It is an age related process, although estrogen deficit also play a very important role in its pathogenesis.

It is estimated that 50% of osteoporosis femur fractures expand to total or partial incapacity and that 20% to 30% of individuals suffering from osteoporosis femur fractures show thromboembolic, circulatory or respiratory complications, leading to death in the following two years after the fracture. The most common types of fractures in osteoporosis are vertebral, distal radius (Colle's fracture) and ribs. However, femur fractures are the major cause of morbidity, eventually leading to death.

Appreciation of the mechanisms through which osteoporosis develops requires an understanding of bone remodeling, that is, a continuous cycle of destruction and renewal carried out by specific cells. Abnormalities in bone reabsorption or formation constitute the final common pathway through which diverse causes, such as dietary or hormonal insufficiency, can produce bone loss.

Bone turnover is about eight times much faster in the trabecular bone than in cortical one. So, the increase in bone turnover that takes place in the menopausal period will lead to a bone loss especially in sites that are rich in the trabecular bone. This is the reason why vertebral bones are the primary sites of bone loss in osteoporosis.

Remodeling is initiated by hormonal or physical signals that cause mononuclear marrow-derived precursor cells to cluster on the bone surface, were they fuse into multinucleated osteoblasts. This process is mediated by osteoblasts, which release a number of chemical mediators. These, in turn, stimulate the synthesis of various factors that promote the proliferation of hemopoletic cells. In the cortical bone, osteoblasts fuse to form a “cutting cone” that excavates a reabsorption tunnel to form a Harvesian canal. When the osteoclastic reabsorption is finished, bone formation ensues. Local release of chemical mediators, probably TGFb and IGF1, attract pre-osteoblasts that mature into osteoblasts and replace the missing bone by secreting new collagen and other matrix constituents.

So, bone turnover can be seen as a process regulated by a macro system (circulating hormones) integrated into a local micro system (local growth factors, cytokines, etc. . . .).

Reabsorption and formation are complete within eight to twelve weeks, with several additional weeks being required to complete mineralization.

Under normal conditions, there is an equivalence in the action of osteoblasts and osteoclasts, so that the amount of bone reabsorbed is equal to the amount of bone replaced. However, remodeling, like other biologic processes, in not entirely efficient, so that it may result into an imbalance. The accumulation of bone deficits will be detected only after many years, suggesting that age-related bone loss may be a normal, predictable phenomenon beginning just after cessation of linear growth.

Given a normal, slightly negative balance, any stimuli that increases the rate of bone remodeling by having more sites involved in this process, will increase the rate of bone loss. This is seen in thyrotoxicosis or primary hyperparathyroidism.

Other stimuli such as glucocorticoids excess, immobilization, ethanol abuse, smoking and age decrease osteoblastic synthetic activity and thus accelerate bone loss.

Radiologic signs of osteoporosis such as bone rarefaction and vertebral compression are only present when we have a reduction of 30% or more in bone mass, and thus are not useful if the aim is an early diagnosis.

There are now several non invasive methods available to access bone mass with reasonable accuracy and precision. The first one to be used for this purpose was the single photon absorptiometry (SPA). This method is only used in skeletal appendages, because it cannot correct the attenuation caused by soft tissues. As the bone mass on these sites does not correspond to the bone mass in critical areas of fractures, such as the vertebral bones, its applicability is limited.

Several studies made possible the development of another method, called dual photon absorptiometry (DPA), which uses ¹⁵³Gadolineum. This method can correct the contribution of soft tissues and thus made possible the measurement of bone mass in areas of more clinical interest.

The method used nowadays is the dual energy X-ray absorptiometry (DEXA), in which the ¹⁵³Gadolineum was substituted by the X-ray. The advantages include a greater reproducibility, a lower dose of radiation, and better resolution. It is also a non-invasive and low-cost method. The limitation is that it cannot differ osteoporosis from osteomalacia.

Osteopenia is defined as a bone density between I-2.5 SD (Standard Deviation) below the mean density of the bone mass peak. Osteoporosis is defined as a bone density below 2.5 SD. Bone density between O—I SD is considered normal. It is recommended a one year interval in serial densitometries in the monitoring of osteoporotic individuals.

Quantitative computed tomography (QTC) is another method that can be used for the evaluation of bone density, and it separates trabecular trabecular from cortical bone. The high doses of radiation, the high cost and the difficulties to access this method limit its use as a routine test. Ultrasound is another method that has been considered, and it has the advantage of low-cost.

Aside from other osteometabolic diseases, such as renal osteodistrophy, osteoporosis is characterized by only slight increases in bone turnover; so, the evaluation of osteoporosis requires highly sensitive markers.

In general, these substances represent either a metabolite of bone matrix breakdown, such as pyridinoline or have an enzymatic activity related to bone formation, such as alkaline phosphatase. It is thought that these markers, along with densitometric studies, would help the identification of women with rapid loss of bone mass, allowing an earlier diagnosis.

Markers of bone formation include osteocalcin, alkaline phosphatase and type I procollagen extension peptide. All of them are secretory products of osteoblasts during bone matrix synthesis. Of these, the first two are available for clinical use and show correlation with bone formation rate.

Alkaline phosphatase is the most used marker to estimate bone formation, but it is not specific for the bone as it includes other sites of production, such as the liver and small intestine. In the absence of other conditions that interfere with alkaline phosphatase activity, this marker will indirectly represent bone formation.

Several studies showed that osteocalcin is a more sensitive marker than total alkaline phosphatase in determining bone formation.

Markers of bone reabsorption include urinary hydroxiproline and piridinoline, both of which reflect collagen breakdown. Hydroxiproline is an aminoacid essentially unique to collagen and is not catabolized in the body. It is derived from various types of collagen and thus it is not specific of bone tissue. It is neither a sensitive method as it is metabolized in the liver.

Piridinoline and desoxipiridinoline are specific for bone turnover and are not metabolized in vivo, thus having more specificity and sensitivity than hydroxiproline.

The simultaneous study of bone reabsorption and formation by these multiple markers has more applicability than the study an unique marker.

Bone Biopsy studies provide definitive diagnosis of mastocitosis and myeloma and remains the gold standard for excluding osteomalacia. It is an invasive study and should be reserved for patients with unusual, unexplained disorders; for patients in whom myeloma or mastocitosis requires exclusion; for patients in whom osteomalacia is suspected and for patients with post-menopausal osteoporosis who are in serious condition and whose bone turnover markers are inconclusive.

Estrogen replacement therapy is the single most important way to reduce a woman's risk of osteoporosis during and after menopause. Estrogen replacement therapy is not advised for women having or having had breast cancer or uterine cancer.

Therefore, there is a need for new bone-building drugs, for example by using the strategy of identifying some drugs that build up bone to where it's stronger and the risk of fracture is no longer present, and others that maintain it by preventing breakdown.

There is also a need for new method of diagnosis that would be at the same time sensitive, for the early detection of osteoporosis, and specific, to distinguish it from osteopenia.

### SUMMARY OF THE INVENTION

The present invention relates to methods of diagnosis, therapy, and screening of new therapeutic compounds in the field of osteogenesis, based on the differential expression observed for the genes of the invention, represented by SEQ ID NO 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID NO 229, or SEQ ID No 230 to SEQ ID No 234, or SEQ ID No 235 to SEQ ID No 245.

### BRIEF DESCRIPTION OF THE FIGURES

FIGS. 1 to 7 represents the relationship between members of the Wnt-frizzled family, with bone formation, by measurement of alkaline phosphatase (ALP).

FIG. 8 represents the transcriptional regulation of frizzled receptors 1 and 2 and SFRPs 1, 2 and 4 in the time-course of primary calvaria cells osteoblastic maturation. Murine calvaria cells were obtained from neonatal mice 1-2 days after birth by sequential collagenase digestion). Calvaria cells were cultured until 80% confluence (time 0) and proliferation medium was replaced by differentiation medium (aMEM containing 10% FCS, 2 mM glutamine, 50 mg/ml ascorbic acid and 10 mM b-glycerolphosphate. Total RNAs were extracted at using the RNAplus kit provided by Quantum, harvesting cells from culture days 0, 2, 7, 14, and 21. Changes in relative gene expression were assessed by using GeneChips (Affymetrix). Results are expressed in ratios using the time 0 as denominator, significant changes in expression (pval<0.1 and ratios>1.5×) are indicated with **.

FIG. 9 represents the transcriptional regulation of Wnt 's, frizzled receptors and SFRPs during maturation of human bone marrow (BMSC) and trabecular bone (NHBC) purified primary cells. BMSC and NHBC cell populations were harvested and dual labelled for STRO-1 and alkaline phosphatase as described (Stewart, K et al, JBMR 11:P 208 (1996) prior to sorting by flow cytometry. Sort regions were the set within each of the quadrants and cells sorted into four population. Cells recovered were re-analyzed by fow cytometry for purity, counted, then pelleted and stored at −80° C. The STRO—I+fraction correspond to less differentiated osteoblast precussors (R5), the STRO-l+/AP+ to more mature osteoblasts (R3) and the AP+ fraction to mature osteoblasts (R2). Total RNAs were extracted at using the RNAplus kit provided by Quantum. Changes in relative gene expression were assessed by using GeneChips (Affynmetrix). Results are expressed in ratios using the time 0 as denominator, significant changes in expression (pval<0.1 and ratios>1.5×) are indicated with **, ratios>1.5 with pval<0.15 are indicated with *.

FIG. 10 represents the effect of BMP2 on the expression of Gas6 and Ufo/Ax1 in pluripotential mensenchymal cell lines C3H10T1/2 and C2C1/2 cells and the osteoblast-like cells MC3T3-E1 were cultured in the presence or absence of 100 ng of recombinant BMP2 for 4 (4d) or 3 (3d) days. Total RNAs were extracted at using the RNAplus kit provided by Quantum. Changes in relative gene expression were assessed by using GeneChips (Affymetrix). Results are expressed in ratios using the untreated cells values as denominators, significant changes in expression (pval<0.1 and ratios>1.5x) are indicated with **.

FIG. 11 represents the transcriptional regulation of gas6 and Ufo/Ax1 in the time-course of primary calvaria cells osteoblastic maturation. Murine calvaria cells were obtained from neonatal mice 1-2 days after birth by sequential collagenase digestion and were cultured until 80% confluence (time 0), proliferation medium was replaced by differentiation medium (aMEM containing 10% FCS, 2 mM glutamine, 50 mg/ml ascorbic acid and 10 mM b-glycerolphosphate). Total RNAs were extracted, harvesting cells from culture days 0, 2, 7, 14, and 21. Changes in relative gene expression were assessed by using GeneChips (Affymetrix). Results are expressed in ratios using the time 0 as denominator, significant changes in expression (pval<0.1 and ratios>1.5×) are indicated with **.

FIG. 12 represents the transcriptional regulation of CCN family and LRP receptors in the time-course of primary calvaria cells osteoblastic maturation. Murine calvaria cells were obtained from neonatal mice 1-2 days after birth by sequential collagenase digestion and were cultured until 80% confluence (time 0), proliferation medium was replaced by differentiation medium (aMEM containing 10% FCS, 2 mM glutamine, 50 mg/ml ascorbic acid and 10 mM b-glycerolphosphate). Total RNAs were extracted, harvesting cells from culture days 0, 2, 7, 14, and 21. Changes in relative gene expression were assessed by using GeneChips (Affymetrix). Results are expressed in ratios using the time 0 as denominator.

### DESCRIPTION OF THE INVENTION

Therefore the present invention relates to a method of diagnosis of osteoporosis in a patient, which method comprises analyzing gene expression of at least one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID NO 211 to SEQ ID No 229, or SEQ ID No 230 to SEQ ID No 234, or SEQ ID No 235 to SEQ ID No 245, in a sample obtained from said patient.

It is noted that, in this description, the termn “one of SEQ ID No 1 to SEQ ID No 150” is identical to “chosen from the group consisting of SEQ ID No 1, SEQ ID No 2, . . . , SEQ ID No 149, SEQ ID No 150” and that all sequences are thus individually singled out, the chosen writing being made to lighten the description.

The expression of the genes represented by SEQ ID No 1 to SEQ ID No 150, or SEQ ID No 151, 152 or 153, or SEQ ID No 154 to SEQ ID No 196, or SEQ ID NO 197 to SEQ ID No 210, or SEQ ID No 211 to SEQ ID No 229, or SEQ ID No 230 to SEQ ID No 234, or SEQ ID No 235 to SEQ ID No 245 may be analyzed by various methods known form the person skilled in the art. In a preferred embodiment, said gene expression analysis is performed by the steps of making complementary DNA (cDNA) from messenger RNA (mRNA) in the sample, optionally amplifying portions of the cDNA corresponding to at least one of SEQ ID NO 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID NO 229, or SEQ ID No 230 to SEQ ID No 234, or SEQ ID No 235 to SEQ ID No 245, and detecting the cDNA optionally amplified, thereby diagnosing osteoporosis.

It is often advantageous to amplify the cDNA obtained after reverse transcription, as this step gives the possibility to label said cDNA, especially by using labeled (especially radioactive or fluorescent) primers or nucleotides. This also helps in the detection of low represented species of mRNA.

In one embodiment, such an analysis is performed on one or more genes chosen SEQ ID No 1 to SEQ ID No 150, or SEQ ID No 151, 152 or 153, or SEQ ID NO 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, and the multiplex analysis is preferably performed on a DNA chip, that contains, at its surface, probes that are complementary to the cDNA that has been optionally amplified.

In one embodiment, the method of the present invention is performed starting from a sample isolated from a patient, that is from a tissue which is a bone, a cartilaginous tissue, or from blood or other body fluid.

As previously exposed, it is often advantageous, in order to facilitate the detection of the cDNA obtained from the mRNA in the patient sample, to use labeled specific oligonucleotide primer(s) or to use a labeled specific probe. The different labels that may be used are well known to the person skilled in the art, and one can cite ³²P, ³³P, ³⁵S, ³H or ¹²⁵I. Non radioactive labels may be selected from ligants as biotin, avidin, streptavidin, dioxygenin, haptens, dyes, luminescent agents like radioluminescent, chemoluminescent, bioluminescent, fluorescent or phosphorescent agents.

The amplification of the cDNA obtained from the mRNA may be carried out by different techniques, the preferred one being polymerase chain reaction (PCR). It is also possible to use the ligase chain reaction (LCR), the transcription-based amplification system (TAS), the self-sustained sequence replication system (SSR).

As previously exposed, it may be advantageous to detect the cDNA with a DNA chip that contains sequences to which said cDNA may hybridize under stringent conditions. In particular, such a method of analysis is well adapted for detecting, at the same time, large number of the genes represented by SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

Such a DNA chip that harbors at least one probe that hybridizes under stringent conditions with one of SEQ ID No 1 to SEQ ID No 150, or SEQ ID No 151, 152 or 153, or SEQ ID No 154 to SEQ ID No 196, or SEQ ID No 197 to SEQ ID NO 210, or SEQ ID No 211 to SEQ ID No 229, or SEQ ID No 230 to SEQ ID No 234, or SEQ ID No 235 to SEQ ID No 245 is also an object of the present invention.

It is to be noted that a probe according to the present invention has to be understood accordingly to the art, and represents in particular a nucleic acid that has between about 15 to 150, more preferably about 25 to 100, more preferably about 30 to 75, more preferably about 40 to 60, more preferably about 50 bases.

Preferably, the DNA chip according to the invention harbors probes that hybridize with at least 5, preferably at least 10, more preferably at least 25, even more preferably at least 30, even more preferably at least 45, even more preferably at least 60 sequences chosen in SEQ ID No 1 to SEQ ID No 196. Preferably the probes hybridize with the human sequences, that are chosen in SEQ ID No 76 to SEQ ID No 196.

As used herein, the term “hybridizes under stringent conditions” is intended to describe conditions for hybridization and washing under which the nucleotide sequences having at least 60%, 65%, 70%, 75% and preferably 80% or 90%, or 95% or greater identity to each other typically remain hybridized to each other.

The stringent hybridization conditions may be defined as described in Sambrook et al. ((1989) Molecular cloning: a laboratory manual. 2^{nd} Ed. Cold Spring Harbor Lab., Cold Spring Harbor, N.Y.), with the following conditions: 5× or 6×SCC, 60° C. Highly stringent conditions that can also be used for hybridization are defined with the following conditions: 6×SSC, 65° C.

Hybridization ADN-ADN or ADN-ARN may be performed in two steps: (1) prehybridization at 42° C. pendant 3 h in phosphate buffer (20 mM, pH 7.5) containing 5 or 6×SSC (1×SSC corresponding to a solution 0.15 M NaCl+0.015 M sodium citrate), 50% formamide, 7% sodium dodecyl sulfate (SDS), 10× Denhardt's, 5% dextran sulfate et 1% salmon sperm DNA; (2) hybridization during up to 20 at a temperature of 60 or 65° C. followed by different washes (about 20 minutes at in 2×SSC+2% SDS, then 0.1×SSC+0.1% SDS). The last wash is performed in 0.1×SSC+0.1% SDS for about 30 minutes at about 60-65° C. this high stringency hybridization conditions may be adapted by a person skilled in the art.

Another alternative for the analyze of the presence of the cDNAs is to size separate them by electrophoresis prior to detection, and then perform a blotting and autoradiography on the separated cDNA. It is also possible to perform (a) dot blot(s), with (a) specific probe(s), to avoid the step of separation of the cDNAs.

These methods are preferred in particular for the detection of a low number of cDNAs.

It is also possible to detect directly the mRNA obtained from cells out of said sample, in particular by carrying out a Northern Blot.

The inventors of the present application have demonstrated that the genes represented by SEQ ID No 1 to SEQ ID No 150 are differentially expressed in models of osteogenesis, some of them being upregulated, and others down-regulated, upon being put in contact with a stimulator of osteogenesis.

In a preferred embodiment, one detects genes that are upregulated upon osteogenesis and that are represented by one of SEQ ID No 1 to SEQ ID No 9, SEQ ID No 11 to 20, SEQ ID No 27, SEQ ID No 33 to 36, SEQ ID No 45 to 50, SEQ ID NO 53, SEQ ID No 54, SEQ ID No 58 to 62, SEQ ID No 66, SEQ ID No 69 to 75, SEQ ID No 76 to SEQ ID No 84, SEQ ID No 86 to 95, SEQ ID No 102, SEQ ID No 108 to 111, SEQ ID No 120 to 125, SEQ ID No 128, SEQ ID No 129, SEQ ID No 133 to 137, SEQ ID No 141, SEQ ID No 144 to 150, SEQ ID No 156, SEQ ID No 158 to SEQ ID No 161, SEQ ID No 164 to SEQ ID No 167, SEQ ID No 170 to SEQ ID No 174, SEQ ID No 176, SEQ ID No 177, SEQ ID No 178, SEQ ID No 180 to SEQ ID No 185, SEQ ID No 187, SEQ ID No 191 to SEQ ID No 194, SEQ ID No 196.

In another embodiment, one detects genes that are downregulated upon osteogenesis and that are represented by one of SEQ ID NO 10, SEQ ID No 21 to 26, SEQ ID No 28 to 32, SEQ ID No 37 to 44, SEQ ID No 51, SEQ ID No 52, SEQ ID No 55 to 57, SEQ ID No 63 to 65, SEQ ID No 67, SEQ ID No 68, SEQ ID No 85, SEQ ID No 96 to 101, SEQ ID No 103 to 107, SEQ ID No 112 to 119, SEQ ID No 126, SEQ ID No 127, SEQ ID No 130 to 132, SEQ ID No 138 to 140, SEQ ID No 142, SEQ ID No 143, SEQ ID No 154, SEQ ID No 155, SEQ ID No 157, SEQ ID No 162, SEQ ID No 163, SEQ ID No 168, SEQ ID No 196, SEQ ID No 175, SEQ ID No 176, SEQ ID No 179, SEQ ID No 186, SEQ ID No 188, SEQ ID No 189, SEQ ID No 190, SEQ ID No 195.

In yet another embodiment, the present invention relates to a nucleic acid molecule that hybridizes under stringent conditions to one or more of the nucleic acid sequences (SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245) noted above that are either upregulated or down-regulated during osteogenesis.

In order to diagnose osteoporosis, it is therefore interesting to detect variations in the expression level of said genes in tissues of a patient.

Therefore the present invention relates to another method of diagnosis of osteoporosis in a mammal comprising the steps of:

a) contacting a sample of mammalian bone or cartilaginous tissue with an agent for specifically detecting endogenous expression of one of SEQ ID NO 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 in said tissue,

b) detecting a level of endogenous expression of said gene in said tissue; and

c) comparing said level of endogenously expressed gene represented by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 23.0 to SEQ ID No 234, or SEQ ID No 235 to SEQ ID No 245 in said tissue with a reference level of said gene represented by one of SEQ ID No 1 to SEQ ID NO 150, or SEQ ID No 151, 152 or 153, or SEQ ID No 154 to SEQ ID NO 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 endogenously expressed in undiseased mammalian bone or cartilaginous tissue to diagnose osteoporosis in said mammal.

In one embodiment, said agent used for the specific detection of endogenous expression of said gene is a nucleic acid probe that hybridizes specifically with RNA transcribed from said gene chosen from SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 present in cells of said tissue, or cDNA obtainable from said RNA.

In another embodiment, said agent is a monoclonal or polyclonal antibody that specifically recognizes the protein or peptide sequence coded by said gene chosen from SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, or by a gene chosen from the genes hybridizing under stringent conditions to one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

In order to obtain an accurate result when determining the differential expression of one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, it may be useful to use an internal standard, and to complete the above-developed method of the invention with the following additional steps of:

d) contacting a sample of said mammalian bone or cartilaginous tissue with a control nucleic acid probe that hybridizes specifically with RNA transcribed from a gene expressed uniformly in mammalian tissues;

e) detecting a level of expression of said gene in said tissue; and

f) comparing the relative expression levels of said gene represented by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID NO 234, or SEQ ID No 235 to SEQ ID No 245 and said gene in said tissue, with the relative expression levels of said gene represented by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 and said gene in undamaged or undiseased mammalian bone or cartilaginous tissue.

Indeed, it is essential to ensure that about the same quantity of starting material is used for the comparison between the test sample and the standard (undiseased) sample. The analysis of the quantity of mRNA from a gene that is uniformly expressed will respond to this concern. This will allow to reduce the variability and uncertainty obtained when performing the quantitative analysis of differential expression of the genes disclosed in the invention.

In one embodiment, said gene expressed uniformly in mammalian tissues is actin. The present invention discloses the sequences of genes that are shown to be differentially expressed in the phenomenon of osteogenesis. Some of the genes are over-expressed while others are under-expressed during this complex process.

Using this data, the present invention relates to a method method for promoting osteogenesis and/or preventing osteoporosis comprising administering to a subject a therapeutically effective amount of a protein product coded by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, wherein said protein product promotes osteogenesis and/or prevents osteoporosis.

In a preferred embodiment, said protein is a secreted protein, and is coded by one of SEQ ID No 1 to 26, SEQ ID No 71, SEQ ID No 76 to SEQ ID No 101, SEQ ID No 154 to SEQ ID No 166.

In another preferred embodiment, said protein is a membrane associated enzyme, and is coded by one of SEQ ID No 27 to 30, 102 to 105, SEQ ID No 167, SEQ ID No 168 or SEQ ID No 169.

In another preferred embodiment, said protein is a membrane associated/putative receptor, and is coded by one of SEQ ID No 31 to 44, 74, 106 to 119, and 149, SEQ ID No 152, SEQ ID No 170 to SEQ ID No 174.

In another preferred embodiment, said protein is a receptor GPCR, an ion channel or a transporter, and is coded by one of SEQ ID No 45 to 52, 120 to 127, SEQ ID No 175 to SEQ ID No 181.

In another preferred embodiment, said protein is an intracellular enzyme and is coded by one of SEQ ID No 53 to 57, 128 to 132, SEQ ID No 182, SEQ ID No 183 or SEQ ID No 184.

In another preferred embodiment, said protein is a transcription factor or an orphan nuclear receptor and is coded by one of SEQ ID No 58 to 64, 69, 133 to 139, 144, SEQ ID No 151, SEQ ID No 185 to SEQ ID No 192.

In another preferred embodiment, said protein is involved in intracellular signal transduction and is coded by one of SEQ ID No 65 to 68, and 140 to 143, SEQ ID No 153, SEQ ID No 193 to SEQ ID No 196.

In another preferred embodiment, said protein is coded by one of SEQ ID No 70, 72, 73, 75, 145, 146, 147, 148, and 150.

In another preferred embodiment, said protein is part of the Wnt-frizzled family, and is preferably coded by one of SEQ ID No 197 to SEQ ID No 210.

In another preferred embodiment, said protein is part of the Ephin-ephrin family and is preferably coded by one of SEQ ID No 211 to SEQ ID No 229.

In another preferred embodiment, said protein is part of the Tyro3 family and is preferably coded by one of SEQ ID No 230 to SEQ ID No 234.

In another preferred embodiment, said protein is part of the CCN family and is preferably coded by one of SEQ ID No 235 to SEQ ID No 245.

In another preferred embodiment, the expression of said protein is upregulated upon osteogenesis.

In another embodiment, the expression of said protein is down-regulated upon osteogenesis. The invention also relates to a method for promoting osteogenesis and/or preventing osteoporosis comprising administering to a subject a therapeutically effective amount of a nucleic acid comprising one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, wherein said nucleic acid product promotes osteogenesis and/or prevents osteoporosis.

In a preferred embodiment, said nucleic acid codes for a secreted protein, and is one of SEQ ID No 1 to 26, SEQ ID No 71, SEQ ID No 76 to SEQ ID No 101, SEQ ID No 154 to SEQ ID No 166.

In another preferred embodiment, said nucleic acid codes for a membrane associated enzyme, and is one of SEQ ID No 27 to 30, 102 to 105, SEQ ID No 167, SEQ ID No 168 or SEQ ID No 169.

In another preferred embodiment, said nucleic acid codes for a membrane associated/putative receptor, and is one of SEQ ID No 31 to 44, 74, 106 to 119, and 149, SEQ ID No 152, SEQ ID No 170 to SEQ ID No 174.

In another preferred embodiment, said nucleic acid codes for a receptor GPCR, an ion channel or a transporter, and is one of SEQ ID No 45 to 52, 120 to 127, SEQ ID No 175 to SEQ ID No 181.

In another preferred embodiment, said nucleic acid codes for an intracellular enzyme and is one of SEQ ID No 53 to 57, 128 to 132, SEQ ID No 182, SEQ ID No 183 or SEQ ID No 184.

In another preferred embodiment, said nucleic acid codes for a transcription factor or an orphan nuclear receptor and is one of SEQ ID No 58 to 64, 69, 133 to 139, 144, SEQ ID No 151, SEQ ID NO 185 to SEQ ID No 192.

In another preferred embodiment, said nucleic acid codes for a protein involved in intracellular signal transduction and is one of SEQ ID No 65 to 68, and 140 to 143, SEQ ID No 153, SEQ ID No 193 to SEQ ID No 196.

In another preferred embodiment, said nucleic acid is one of SEQ ID No 70, 72, 73, 75, 145, 146, 147, 148, and 150.

In another preferred embodiment, said acid nucleic codes for a protein that is part of the Wnt-frizzled family, and is preferably one of SEQ ID No 197 to SEQ ID No 210.

In another preferred embodiment, said acid nucleic codes for a protein that is part of the Ephin-ephrin family and is preferably one of SEQ ID No 211 to SEQ ID No 229.

In another preferred embodiment, said acid nucleic codes for a protein that is part of the Tyro3 family and is preferably one of SEQ ID No 230 to SEQ ID No 234.

In another preferred embodiment, said acid nucleic codes for a protein that is part of the CCN family and is preferably one of SEQ ID No 235 to SEQ ID No 245.

In another preferred embodiment, the expression of said nucleic acid is upregulated upon osteogenesis.

In another embodiment, the expression of said nucleic acid is down-regulated upon osteogenesis.

In a preferred embodiment, said nucleic acid is administered to said subject such as to enter osteoblastic or osteoclastic cells, that is the cells that are play an important part in osteogenesis and bone remodelling.

For penetration of the nucleic acid within the cells, different means may be used by the person skilled in the art. In particular, it is possible to introduce said nucleic acid within the cells by means of a viral vector.

Said virus may be of human or of non-human origin, as long as it possesses the capability to infect the cells of the patient. In particular, said virus is chosen from the group consisting of adenoviridiae, retroviridiae (oncovirinae such as RSV, spumavirinae, lentivirus), poxyiridiae, herpesviridiae (HSV, EBV, CMV . . . ), iridiovirus, hepadnavirus (hepatitis B virus), papoviridiae (SV40, papillomavirus), parvoviridiae (adeno-associated virus . . . ), reoviridiae (reovirus, rotavirus), togaviridiae (arbovirus, alphavirus, flavivirus, rubivirus, pestivirus), coronaviriadiae, paramyxoviridae, orthomixoviridae, rhabdoviridae (rabies virus), bunyaviridae, arenaviridae, picomaviridae (enterovirus, Coxsackievirus, echovirus, rhinovirus, aphtovirus, cardiovirus, hepatitis A virus . . . ), Modified Virus Ankara, and derived viruses thereof.

By derived viruses, it is intended to mean that the virus possesses modifications that adapt it to the human being (if it is a virus from a non-human origin that could not infect human cells without said modifications), and/or that reduce its potential or actual pathogenicity. In particular, it is best if the virus used for the gene transfer is defective for replication within the human body. This is an important safety concern, as the control of the expression of the functional gene may be a concern for the implementation of the method of the invention. One does not either whish to have a dissemination to other cells or to other people of the viral vector carrying the gene of therapeutic interest.

This is why the viral vector used in the method of the invention is preferably deficient for replication, and would therefore be prepared with the help of a auxiliary virus or in a complementary cell line, that would bring in trans the genetic material needed for the preparation of a sufficient viral titer.

Such defective viruses and appropriate cell lines are described in the art, for example in U.S. Pat. No. 6,133,028 that describes deficient adeno-associated viruses (AAV) and the associated complementation cell lines, and the content of which is herein incorporated by reference. Other suitable viruses are described for example in WO 00/34497. For adenoviruses or AAV, it may be interesting to delete the E1 and/or E4 regions.

For the MFG virus described below, one can use the complementation T-CRIP cell line that was described in Hacein-Bey et al. (1996, Blood. 87, 3108-16), incorporated herein by reference. Other appropriate cell lines could also be used. In order to improve the long lasting effect of the correction, one would prefer a virus that allows the integration of said functional gene into a chromosome of the infected cells.

In particular, one would chose adenoviruses, some of which defective for replication are well know by the person skilled in the art, or retroviruses, in particular murine derived retroviruses. Among the retroviruses that can be used, one would prefer a myeloproliferative sarcoma virus (MPSV)-based vector as described in Bunting et al. (1998, Nature Medecine, 4, 58-64, the content of which is incorporated herein by reference). Another well suited retrovirus that can be used for the implementation of the method of the invention is the MFG vector, derived from the MLV virus (Moloney retrovirus), described in Hacein-Bey et al. (1996, Blood. 87, 3108-16) or Cavazzana-Calvo et al. (2000, Science, 288, 669-72), the content of both these documents being incorporated herein by reference.

The choice of the virus to be used for the implementation of the method of the invention will be function of the characteristics of said virus and of the complementation cell line. It is clear that different viruses have different properties (in particular LTR in retroviruses), and that the viruses and cell lines cited above are only examples of means that can be used for the implementation of the method of the invention, and that they shall not be considered as restrictive. The person skilled in the art knows how to choose the best combination gene—virus—cell line and/or auxiliary virus for any given situation.

In another embodiment, said nucleic acid is introduced within cells by means of a synthetic vector which can be chosen from the group consisting of a cationic amphiphile, a cationic lipid, a cationic or neutral polymer, a protic polar compound such as propylene glycol, polyethylene glycol, glycerol, ethano, I-methyl-L-2-pyrrolidone or their derivatives, and an aprotic polar compound such as dimethyl sulfoxide (DMSO), diethyl sulfoxide, di-n-propyl sulfoxide, dimethyl sulfone, sulfolane, dimethylformamide, dimethylacetamide, tetramethylurea, acetonitrile or their derivatives. The person skilled in the art is aware of synthetic vectors that can be used and allow a high level of transfection, such as Lifofectine and Lipofectamine reagents available from Life Technologies (Bethesda, Md.).

The present invention also relates to a method for promoting osteogenesis and/or preventing osteoporosis comprising administering to a subject a therapeutically effective amount of an inhibitor of a protein product coded by one of SEQ ID No 1 to SEQ ID No 150, or SEQ ID No 151, 152 or 153, or SEQ ID NO 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID NO 245, wherein said inhibitor of said protein product promotes osteogenesis and/or prevents osteoporosis.

In one embodiment, said inhibitor is a monoclonal or polyclonal antibody directed towards said protein product coded by one of SEQ ID No 1 to SEQ ID No 150, or SEQ ID No 151, 152 or 153, or SEQ ID No 154 to SEQ ID No 196, or SEQ IDN 1197 to SEQ ID No 210, or SEQ ID No 211 to SEQ ID No 229, or SEQ ID No 230 to SEQ ID No 234, or SEQ ID No 235 to SEQ ID No 245.

In a preferred embodiment, said protein is a secreted protein, and is coded by one of SEQ ID No 1 to 26, SEQ ID No 71, SEQ ID No 76 to SEQ ID No 101, SEQ ID No 154 to SEQ ID No 166.

In another preferred embodiment, said protein is a membrane associated enzyme, and is coded by one of SEQ ID No 27 to 30, 102 to 105, SEQ ID No 167, SEQ ID No 168 or SEQ ID No 169.

In another preferred embodiment, said protein is a membrane associated/putative receptor, and is coded by one of SEQ ID No 31 to 44, 74, 106 to 119, and 149, SEQ ID No 152, SEQ ID No 170 to SEQ ID No 174.

In another preferred embodiment, said protein is a receptor GPCR, an ion channel or a transporter, and is coded by one of SEQ ID No 45 to 52, 120 to 127, SEQ ID No 175 to SEQ ID No 181.

In another preferred embodiment, said protein is an intracellular enzyme and is coded by one of SEQ ID No 53 to 57, 128 to 132, SEQ ID No 182, SEQ ID No 183 or SEQ ID No 184.

In another preferred embodiment, said protein is a transcription factor or an orphan nuclear receptor and is coded by one of SEQ ID No 58 to 64, 69, 133 to 139, 144, SEQ ID No 151, SEQ ID No 185 to SEQ ID No 192.

In another preferred embodiment, said protein is involved in intracellular signal transduction and is coded by one of SEQ ID No 65 to 68, and 140 to 143, SEQ ID No 153, SEQ ID No 193 to SEQ ID No 196. In another preferred embodiment, said protein is part of the Wnt-frizzled family, and is preferably coded by one of SEQ ID No 197 to SEQ ID No 210.

In another preferred embodiment, said protein is part of the Ephin-ephrin family and is preferably coded by one of SEQ ID No 211 to SEQ ID No 229.

In another preferred embodiment, said protein is part of the Tyro3 family and is preferably coded by one of SEQ ID No 230 to SEQ ID No 234.

In another preferred embodiment, said protein is part of the CCN family and is preferably coded by one of SEQ ID No 235 to SEQ ID No 245.

In another preferred embodiment, said protein is coded by one of SEQ ID NO 70, 72, 73, 75, 145, 146, 147, 148, and 150.

In another preferred embodiment, the expression of said protein is upregulated upon osteogenesis.

In another embodiment, the expression of said protein is down-regulated upon osteogenesis.

In another embodiment, said inhibitor is a ribozyme that leads to degradation of the mRNA corresponding to an nucleic acid represented by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

In another embodiment, said inhibitor is a nucleic acid, antisense to the nucleic acid represented by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

Such a nucleic acid may be a deoxyribonucleotide or ribonucleotide polymer in single-stranded form, and encompasses known analogues of natural nucleotides that hybridize to nucleic acids in a manner similar to naturally occurring nucleotides. Known and preferred analogues include polymers of nucleotides with phosphorothioate or methylphosphonate liaisons, or peptide nucleic acids.

In a preferred embodiment, said nucleic acid target of the inhibitor codes for a secreted protein, and is one of SEQ ID No 1 to 26, SEQ ID No 71, SEQ ID No 76 to SEQ ID No 101, SEQ ID No 154 to SEQ ID No 166.

In another preferred embodiment, said nucleic acid target of the inhibitor codes for a membrane associated enzyme, and is one of SEQ ID No 27 to 30, 102 to 105, SEQ ID No 167, SEQ ID No 168 or SEQ ID No 169.

In another preferred embodiment, said nucleic acid target of the inhibitor codes for a membrane associated/putative receptor, and is one of SEQ ID No 31 to 44, 74, 106 to 119, and 149, SEQ ID No 152, SEQ ID No 170 to SEQ ID No 174.

In another preferred embodiment, said nucleic acid target of the inhibitor codes for a receptor GPCR, an ion channel or a transporter, and is one of SEQ ID No 45 to 52, 120 to 127, SEQ ID No 175 to SEQ ID No 181.

In another preferred embodiment, said nucleic acid target of the inhibitor codes for an intracellular enzyme and is one of SEQ ID No 53 to 57, 128 to 132, SEQ ID No 182, SEQ ID No 183 or SEQ ID No 184.

In another preferred embodiment, said nucleic acid target of the inhibitor codes for a transcription factor or an orphan nuclear receptor and is one of SEQ ID NO 58 to 64, 69, 133 to 139, 144, SEQ ID No 151, SEQ ID No 185 to SEQ ID No 192.

In another preferred embodiment, said nucleic acid target of the inhibitor codes for a protein involved in intracellular signal transduction and is one of SEQ ID NO 65 to 68, and 140 to 143, SEQ ID No 153, SEQ ID No 193 to SEQ ID No 196.

In another preferred embodiment, said acid nucleic target of the inhibitor codes for a protein that is part of the Wnt-frizzled family, and is preferably one of SEQ ID No 197 to SEQ ID No 210.

In another preferred embodiment, said acid nucleic target of the inhibitor codes for a protein that is part of the Ephin-ephrin family and is preferably one of SEQ ID No 211 to SEQ ID No 229.

In another preferred embodiment, said acid nucleic target of the inhibitor codes for a protein that is part of the Tyro3 family and is preferably one of SEQ ID No 230 to SEQ ID No 234.

In another preferred embodiment, said acid nucleic target of the inhibitor codes for a protein that is part of the CCN family and is preferably one of SEQ ID NO 235 to SEQ ID No 245.

In another preferred embodiment, said nucleic acid target of the inhibitor is In another preferred embodiment, said protein is coded by one of SEQ ID No 70, 72, 73, 75, 145, 146, 147, 148, and 150.

In another preferred embodiment, the expression of said nucleic acid target of said inhibitor is upregulated upon osteogenesis.

In another embodiment, the expression of said nucleic acid target of said inhibitor is downregulated upon osteogenesis.

Said nucleic acid that is a inhibitor of the nucleic acid chosen from at least one of SEQ ID No 1 to SEQ ID No 150, or SEQ ID No 151, 152 or 153, or SEQ ID NO 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 may be introduced in the same kind of cells, using the same vectors as already described above.

The present invention also relates to the use of

a protein product coded by one of SEQ ID No 1 to SEQ ID No 150, or SEQ ID No 151, 152 or 153, or SEQ ID No 154 to SEQ ID NO 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, wherein said protein product promotes osteogenesis and/or prevents osteoporosis,

a nucleic acid comprising one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, wherein said nucleic acid product promotes osteogenesis and/or prevents osteoporosis

an inhibitor of a protein product coded by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, wherein said inhibitor of said protein product promotes osteogenesis and/or prevents osteoporosis

for the preparation of a medicament intended for the treatment of osteoporosis and/or the promotion of osteogenesis.

The present invention thus relates to a method of therapy and/or prevention of osteoporosis, based on the nucleic acid sequences and/or protein products identified by the inventors of this application as being up or down-regulated in osteogenesis. An other aspect of the invention relates to the use of these sequences and proteins in methods of detection, identification, and/or screening of new compounds useful for the treatment of bone diseases, especially the treatment and/or prevention of osteoporosis, and for osteogenesis.

Thus, the present invention relates to a method for detecting, identifying and/or screening a compound having a role in osteogenesis, comprising the steps of:

a) bringing said compound in contact with a cell model of osteogenesis, and

b) comparing the level of expression of one of SEQ ID No 1 to SEQ ID NO 150, or SEQ ID No 151, 152 or 153, or SEQ ID No 154 to SEQ ID No 196, or SEQ ID No 197 to SEQ ID No 210, or SEQ ID NO 211 to SEQ ID No 229, or SEQ ID No 230 to SEQ ID No 234, or SEQ ID No 235 to SEQ ID No 245 in said cell model with regard to said level of expression of said gene in the same model to which said compound has not been brought in contact,

the role of said compound in osteogenesis being deduced from the presence of a difference between said levels of expression between the two systems.

In another aspect, the invention relates to a method for detecting, identifying and/or screening a compound useful for modulation of osteogenesis, comprising the steps of:

a) bringing said compound in contact with a protein coded by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, and

b) analyzing the interaction between said compound and said protein, the utility of said compound in the modulation of osteogenesis being deduced from the presence of an interaction between said compound and said protein coded by one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

In a preferred embodiment, one detects genes that are upregulated upon osteogenesis and that are represented by one of SEQ ID No 1 to SEQ ID No 9, SEQ ID NO Il to 20, SEQ ID No 27, SEQ ID No 33 to 36, SEQ ID No 45 to 50, SEQ ID No 53, SEQ ID No 54, SEQ ID No 58 to 62, SEQ ID No 66, SEQ ID No 69 to 75, SEQ ID No 76 to SEQ ID No 84, SEQ ID No 86 to 95, SEQ ID No 102, SEQ ID No 108 to 111, SEQ ID No 120 to 125, SEQ ID No 128, SEQ ID No 129, SEQ ID No 133 to 137, SEQ ID No 141, SEQ ID No 144 to 150, SEQ ID No 156, SEQ ID No 158 to SEQ ID No 161, SEQ ID No 164 to SEQ ID No 167, SEQ ID No 170 to SEQ ID No 174, SEQ ID No 176, SEQ ID No 177, SEQ ID No 178, SEQ ID No 180 to SEQ ID No 185, SEQ ID No 187, SEQ ID No 191 to SEQ ID No 194, SEQ ID No 196.

In another embodiment, one detects genes that are downregulated upon osteogenesis and that are represented by one of SEQ ID No 10, SEQ ID No 21 to 26, SEQ ID No 28 to 32, SEQ ID No 37 to 44, SEQ ID No 51, SEQ ID No 52, SEQ ID No 55 to 57, SEQ ID No 63 to 65, SEQ ID No 67, SEQ ID No 68, SEQ ID No 85, SEQ ID No 96 to 101, SEQ ID No 103 to 107, SEQ ID No 112 to 19, SEQ ID No 126, SEQ ID No 127, SEQ ID No 130 to 132, SEQ ID No 138 to 140, SEQ ID No 142, SEQ ID No 143, SEQ ID No 154, SEQ ID No 155, SEQ ID No 157, SEQ ID No 162, SEQ ID No 163, SEQ ID No 168, SEQ ID No 196, SEQ ID No 175, SEQ ID No 176, SEQ ID No 179, SEQ ID No 186, SEQ ID No 188, SEQ ID No 189, SEQ ID No 190, SEQ ID No 195.

In a preferred embodiment, said protein is a secreted protein, and is coded by one of SEQ ID No 1 to 26, SEQ ID No 71, SEQ ID No 76 to SEQ ID No 101, SEQ ID No 154 to SEQ ID No 166.

In another preferred embodiment, said protein is a membrane associated enzyme, and is coded by one of SEQ ID No 27 to 30, 102 to 105, SEQ ID No 167, SEQ ID No 168 or SEQ ID No 169.

In another preferred embodiment, said protein is a membrane associated/putative receptor, and is coded by one of SEQ ID No 31 to 44, 74, 106 to 119, and 149, SEQ ID No 152, SEQ ID No 170 to SEQ ID No 174.

In another preferred embodiment, said protein is a receptor GPCR, an ion channel or a transporter, and is coded by one of SEQ ID No 45 to 52, 120 to 127, SEQ ID No 175 to SEQ ID No 181.

In another preferred embodiment, said protein is an intracellular enzyme and is coded by one of SEQ ID No 53 to 57, 128 to 132, SEQ ID No 182, SEQ ID No 183 or SEQ ID No 184.

In another preferred embodiment, said protein is a transcription factor or an orphan nuclear receptor and is coded by one of SEQ ID No 58 to 64, 69, 133 to 139, 144, SEQ ID No 151, SEQ ID No 185 to SEQ ID No 192.

In another preferred embodiment, said protein is involved in intracellular signal transduction and is coded by one of SEQ ID No 65 to 68, and 140 to 143, SEQ ID No 153, SEQ ID No 193 to SEQ ID No 196.

In another preferred embodiment, said protein is coded by one of SEQ ID No 70, 72, 73, 75, 145, 146, 147, 148, and 150.

The present invention thus allows the detection, identification and/or screening of compounds that may be useful for the treatment of osteoporosis. Nevertheless, the compounds identified by one of the methods according to the invention, in order to be used in a therapeutic treatment, may need to be optimized, in order to have a superior activity and/or a lesser toxicity.

Indeed, the development of new drugs is often performed on the following basis:

screening of compounds with the sought activity, on a relevant model, by an appropriate method,

selection of the compounds that have the required properties from the first screening test (here, modulation of osteogenesis),

determination of the structure (in particular the sequence (if possible the tertiary sequence) if they are peptides, proteins or nucleic acids, formula and backbone if they are chemical compounds) of the selected compounds,

optimization of the selected compounds, by modification of the structure (for example, by changing the stereochemical conformation (for example passage of the amino acids in a peptide from L to D), addition of substituants on the peptidic or chemical backbones, in particular by grafting groups or radicals on the backbone, modification of the peptides (se in particular Gante “Peptidomimetics”, in Angewandte Chemie-International Edition Engl. 1994, 33. 1699-1720),

passage and screening of the “optimized” compounds on appropriate models that are often models nearer to the studied pathology. At this stage, one would often use animal models, in particular rodents (rats or mice) or dogs or non-human primates, that are good the models of osteoporosis, or that allow the study of osteogenesis by measurement of the increase of bone density in the animals after administration of the compound.

Therefore, the present invention also relates to a method for identifying a compound useful for treatment of osteoporosis, comprising the steps of:

a) performing a method of the invention, as described above,

b) modifying the compound selected in step a),

c) testing the modified compound of step b) in in vitro and/or in vivo models relevant for assessment of osteoporosis,

d) identifying the compound having a anti-osteoporosis activity superior than for the compound selected in step a).

Step d) of the preceding method he method of may be replaced and/or completed by a step d′):

d′) identifying the compound having the searched biological effect on osteoporosis, with a reduced toxicity in an animal model than the compound selected in step a).

The present invention also relates to the compounds identified by one of the methods of the invention, especially the compounds that have a role in stimulation of bone formation or bone density increase, and/or that are useful for treatment of osteoporosis.

A compound identified by a method according to the invention may be a compound with a chemical backbone, a lipid, a carbohydrate (sugar), a protein, a peptide, an hybrid compound protein-lipid, protein-carbohydrate, peptide-lipid, peptide carbohydrate, a protein or a peptide on which has been branched different chemical residues.

The foreseen chemical compounds (with a chemical backbone), may contain one or more (up to 3 or 4) cycles, especially aromatic cycles, in particular having from 3 to 8 atoms of carbon, and having all kinds of branched groups (in particular lower alkyl, i.e. having from 1 to 6 atoms of carbon, keto groups, alcohol groups, halogen groups . . . ). The person skilled in the art knows how to prepare different variants of a compound starting from a given backbone by grafting these radicals on said backbone.

These compounds, may be used for the preparation of a medicament, destined for the treatment of bone diseases, in particular osteoporosis, or for the promotion of osteogenesis remodeling of bones, and/or increase of bone density.

The present invention also relates to an isolated nucleic acid sequence upregulated in osteogenesis chosen from the group consisting of:

a) one of SEQ ID No 1 to SEQ ID No 9, SEQ ID No 11 to 20, SEQ ID No 27, SEQ ID No 33 to 36, SEQ ID No 45 to 50, SEQ ID No 53, SEQ ID No 54, SEQ ID No 58 to 62, SEQ ID No 66, SEQ ID NO 69 to 75, SEQ ID No 76 to SEQ ID No 84, SEQ ID No 86 to 95, SEQ ID No 102, SEQ ID No 108 to 111, SEQ ID No 120 to 125, SEQ ID No 128, SEQ ID No 129, SEQ ID No 133 to 137, SEQ ID No 141, SEQ ID No 144 to 150, SEQ ID No 156, SEQ ID No 158 to SEQ ID No 161, SEQ ID No 164 to SEQ ID No 167, SEQ ID No 170 to SEQ ID No 174, SEQ ID No 176, SEQ ID No 177, SEQ ID No 178, SEQ ID No 180 to SEQ ID No 185, SEQ ID No 187, SEQ ID No 191 to SEQ ID No 194, SEQ ID No 196,

b) an isolated and purified nucleic acid comprising the nucleic acid of a)

c) an isolated nucleic acid that specifically hybridizes under stringent conditions to the complement of the nucleic acid of a), wherein said nucleic acid encodes a protein that is upregulated in osteogenesis

d) an isolated nucleic acid having at least 80% homology with the nucleic acid of a), wherein said nucleic acid encodes a protein that is upregulated in osteogenesis

e) a fragment of the nucleic acid of a) comprising at least 15 nucleotides.

In a preferred embodiment, said nucleic acid codes for a secreted protein, and is one of SEQ ID No 1 to 9, 11 to 20, SEQ ID No 71, SEQ ID No 76 to SEQ ID No 84, 86 to 95, SEQ ID No 156, SEQ ID No 158 to SEQ ID No 16.1, SEQ ID No 164 to SEQ ID No 166.

In another preferred embodiment, said nucleic acid codes for a membrane associated enzyme, and is one of SEQ ID No 27 and SEQ ID No 102 SEQ ID No 167.

In another preferred embodiment, said nucleic acid codes for a membrane associated/putative receptor, and is one of SEQ ID No 33 to 36, 74, 108 to 111, and 149, SEQ ID No 152, SEQ ID No 170 to SEQ ID No 174.

In another preferred embodiment, said nucleic acid codes for a receptor GPCR, an ion channel or a transporter, and is one of SEQ ID No 45 to 50, 120 to 125, SEQ ID No 176, SEQ ID No 177, SEQ ID No 178, SEQ ID No 180 to SEQ ID No 181.

In another preferred embodiment, said nucleic acid codes for an intracellular enzyme and is one of SEQ ID No 53, 54, 128 and 129, SEQ ID No 182, SEQ ID No 183 or SEQ ID No 184.

In another preferred embodiment, said nucleic acid codes for a transcription factor or an orphan nuclear receptor and is one of SEQ ID No 58 to 62, 69, 133 to 137, 144, SEQ ID No 151, SEQ ID No 185, SEQ ID No 187, SEQ ID No 191 to SEQ ID No 192.

In another preferred embodiment, said nucleic acid codes for a protein involved in intracellular signal transduction and is one of SEQ ID No 66, and 141, SEQ ID No 153, SEQ ID No 193, SEQ ID No 194, SEQ ID No 196.

In another preferred embodiment, said nucleic acid is one of SEQ ID No 70, 72, 73, 75, 145, 146, 147, 148, and 150.

The invention also relates to an isolated nucleic acid sequence downregulated in osteogenesis, chosen from the group consisting of:

a) one of SEQ ID No 10, SEQ ID No 21 to 26, SEQ ID No 28 to 32, SEQ ID No 37 to 44, SEQ ID No 51, SEQ ID No 52, SEQ ID No 55 to 57, SEQ ID No 63 to 65, SEQ ID No 67, SEQ ID No 68, SEQ ID No 85, SEQ ID No 96 to 101, SEQ ID No 103 to 107, SEQ ID No 112 to 119, SEQ ID No 126, SEQ ID No 127, SEQ ID No 130 to 132, SEQ ID No 138 to 140, SEQ ID No 142, SEQ ID No 143, SEQ ID No 154, SEQ ID No 155, SEQ ID No 157, SEQ ID No 162, SEQ ID No 163, SEQ ID No 168, SEQ ID No 196, SEQ ID No 175, SEQ ID No 176, SEQ ID No 179, SEQ ID No 186, SEQ ID No 188, SEQ ID No 189, SEQ ID No 190, SEQ ID No 195,

b) an isolated and purified nucleic acid comprising the nucleic acid of a)

c) an isolated nucleic acid that specifically hybridizes under stringent conditions to the complement of the nucleic acid of a), wherein said nucleic acid encodes a protein that is upregulated in osteogenesis

d) an isolated nucleic acid having at least 80% homology with the nucleic acid of a), wherein said nucleic acid encodes a protein that is upregulated in osteogenesis

e) a fragment of the nucleic acid of a) comprising at least 15 nucleotides.

In a preferred embodiment, said nucleic acid codes for a secreted protein, and is one of SEQ ID No 10, SEQ ID No 21 to 26, SEQ ID No 85, SEQ ID No 96 to 101, SEQ ID No 154, SEQ ID No 155, SEQ ID No 157, SEQ ID No 162, SEQ ID No 163.

In another preferred embodiment, said nucleic acid codes for a membrane associated enzyme, and is one of SEQ ID No 28 to 30, 103 to 105, SEQ ID No 168, SEQ ID No 169.

In another preferred embodiment, said nucleic acid codes for a membrane associated/putative receptor, and is one of SEQ ID No 31, SEQ ID No 32, SEQ ID NO 37 to 44, SEQ ID No 106, SEQ ID No 107 and SEQ ID No 112 to 119.

In another preferred embodiment, said nucleic acid codes for a receptor GPCR, an ion channel or a transporter, and is one of SEQ ID No 51, 52, 126, 127, SEQ ID No 175, SEQ ID No 176, SEQ ID No 179.

In another preferred embodiment, said nucleic acid codes for an intracellular enzyme and is one of SEQ ID No 55 to 57, 130 to 132.

In another preferred embodiment, said nucleic acid codes for a transcription factor or an orphan nuclear receptor and is one of SEQ ID No 63, 64, 138, and 139, SEQ ID No 186, SEQ ID No 188, SEQ ID No 189, SEQ ID No 190.

In another preferred embodiment, said nucleic acid codes for a protein involved in intracellular signal transduction and is one of SEQ ID No 65, 67, 68, 140, 142, and 143, SEQ ID No 195.

Most preferred nucleic acids according to the invention are SEQ ID No 3, 4, 6, 8, 10, 11, 13, 15, 20 to 23, 27, 31, 32, 33, 35, 37, 40, 43 to 46, 48, 50 to 52, 54, 56, 57, 63 to 75, 78, 79, 81, 83, 85, 86, 88, 90, 95 to 98, 102, 106, 107, 108, 110, 112, 115, 118 to 121, 123, 125 to 127, 129, 131, 132, 138 to 150.

In some embodiments, the invention relates to a acid nucleic, the expression of which is modulated during osteogenesis, and that codes for a protein that is part of the Wnt-frizzled family, and is preferably one of SEQ ID No 197 to SEQ ID No 210.

In another preferred embodiment, the invention relates to a acid nucleic, the expression of which is modulated during osteogenesis, and that codes for a protein that is part of the Ephin-ephrin family and is preferably one of SEQ ID No 211 to SEQ ID No 229.

In another preferred embodiment, the invention relates to a acid nucleic, the expression of which is modulated during osteogenesis, and that codes for a protein that is part of the Tyro3 family and is preferably one of SEQ ID No 230 to SEQ ID No 234.

In another preferred embodiment, the invention relates to a acid nucleic, the expression of which is modulated during osteogenesis, and that codes for a protein that is part of the CCN family and is preferably one of SEQ ID No 235 to SEQ ID No 245.

By isolated and purified nucleic acid of b), it is in particular meant to mean a vector comprising the nucleic acid of a).

The stringent hybridization conditions may be defined as described in Sambrook et al. ((1989) Molecular cloning: a laboratory manual. 2^{nd} Ed. Cold Spring Harbor Lab., Cold Spring Harbor, N.Y.), with the following conditions: 5× or 6×SCC, 60° C. Highly stringent conditions that can also be used for hybridization are defined with the following conditions: 6×SSC, 65° C.

Hybridization ADN-ADN or ADN-ARN may be performed in two steps: (1) prehybridization at 42° C. pendant 3 h in phosphate buffer (20 mM, pH 7;5) containing 5 or 6×SSC (1×SSC corresponding to a solution 0.15 M NaCl+0.015 M sodium citrate), 50% formamide, 7% sodium dodecyl sulfate (SDS), 10× Denhardt's, 5% dextran sulfate et 1% salmon sperm DNA; (2) hybridization during up to 20 at a temperature of 60 or 65° C. followed by different washes (about 20 minutes at in 2×SSC+2% SDS, then 0.1×SSC+0.1% SDS). The last wash is performed in 0.1×SSC+0.1% SDS for about 30 minutes at about 60-65° C. this high stringency hybridization conditions may be adapted by a person skilled in the art.

Two polynucleotides are said to be “identical” or “homologous” if the sequence of nucleotides or amino acid residues, respectively, in the two sequences is the same when aligned for maximum correspondence as described below. The term “complementary to” is used herein to mean that the complementary sequence is identical to all or a specified contiguous portion of a reference polynucleotide sequence. Sequence comparisons between two (or more) polynucleotides or polypeptides are typically performed by comparing sequences of two optimally aligned sequences over a segment or “comparison window” to identify and compare local regions of sequence similarity. Optimal alignment of sequences for comparison may be conducted by the local homology algorithm of Smith and Watemian, Ad. App. Math 2: 482 (1981), by the homology alignment algorithm of Neddleman and Wunsch, J. Mol. Biol. 48:443 (1970), by the search for similarity method of Pearson and Lipman, Proc. Natl. Acad. Sci. (U.S.A.) 85:2444 (1988), by computerized implementation of these algorithms (GAP, BESTFIT, BLAST N, BLAST P, FASTA, and TFASTA in the Wisconsin Genetics Software Package, Genetics Computer Group (GCG), 575 Science Dr., Madison, Wis.), or by inspection. In order to determine the optimal window of alignment, the BLAST program could be used, using matrix BLOSUM 62, or matrices PAM or PAM250.

“Percentage of sequence identity or homology” is determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The nucleic acid of d) presents an homology of at least 80%, more preferably 90%, more preferably 95%, more preferably 98%, the most preferable being 99% with the nucleic acid of a).

The fragment of the nucleic acid of a) contain at least 15 bases, more preferably 25, 50, 75, 100, 150, 200, 300 bases. This fragments may be used as primers for amplification, or as probes especially when looking for homologous DNA or DNA hybridizing with the nucleic acid of a). These fragments may be labeled as described above.

The invention also relates to an isolated protein or peptide coded by a nucleic acid of the invention. These proteins or peptides can be obtained after cloning the nucleic acid of the invention in an expression vector, that contains the elements that are necessary for the expression of said protein or peptide in a host cell (prokaryotic or eucaryotic). Such an expression vector may also contain the elements allowing secretion of the protein or peptide. An host cell containing such an expression vector is also an object of the invention.

The expression vectors of the invention contain preferably a promoter, traduction initiation and termination signals, as well as appropriate regions for regulating transcrition. They need to be maintained in the host cell. The person skilled in the art is aware of such vectors and of the ways to produce and purify proteins, especially by using labels (like Histidine Tag, or glutathione). It is also possible to use in vitro translation kits that are widely available, to produce the protein or peptide according to the invention.

The invention also relates to monoclonal or polyclonal antibodies that specifically recognize the protein or peptide of the invention, as well as their fragments, chimeric antibodies, immunoconjugates.

Specific polyclonal antibodies may be obtained from the serum of an animal that has been immunized by a protein or a peptide according to the invention, optionally using an appropriate adjuvant.

Specific monoclonal antibodies may be obtained by the hybridoma culture method described by Köhler et Milstein (1975 Nature 256, 495).

The antibodies according to the invention are, for example, chimeric antibodies, humanized antibodies, Fab ou F(ab′)₂ fragments. They may be immunoconjugates or labeled antibodies.

The antibodies of the invention are well suited for the diagnosis and therapeutic methods of the invention.

The invention also relates to a pharmaceutical composition comprising an pharmaceutically acceptable excipient with at least one of a compound of the invention, a nucleic acid of the invention, a protein of the invention, an antibody of the invention. Appropriate excipients are well known of the person skilled in the art for such a purpose.

The invention also relates to a method for the therapy of a bone disease, especially osteoporosis, or to a method for increasing bone density and/or promoting osteogenesis, comprising administering to a subject one of a compound according to the invention, a nucleic acid according to the invention a protein according to the invention, an antibody according to the invention, and/or a pharmaceutical composition according to the invention.

The invention also relates to the use of a compound according to the invention, especially having an anti-osteoporosis activity, a nucleic acid according to the invention, a protein according to the invention, an antibody according to the invention, and/or a pharmaceutical composition according to the invention, for the manufacture of a medicament for the treatment of a bone disease, especially osteoporosis, or for increasing bone density and/or promoting osteogenesis

The present invention also relates to the determination of the binding partners of the proteins coded by on of SEQ ID No 1 to SEQ ID No 150, or SEQ ID NO 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID NO 234, or SEQ ID No 235 to SEQ ID No 245 by using the double hybrid assay, as described by Finley and Brent (Interaction trap cloning with yeast, 169-203, in DNA Cloning, Expression Systems: a practical Approach, 1995, Oxford Universal Press, Oxford, the content of which is incorporated herein by reference).

Basically, a yeast strain is transformed by two plasmids encoding either the bait protein (the protein coded by one of SEQ ID No 1 to SEQ ID No 150, or SEQ ID NO 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245) or the protein supposed to be a binding partner of the bait protein (the prey protein).

Upon binding of the 2 proteins, a reporting gene is induced and the yeast becomes able to metabolize a substrate in the medium. It is thus possible to determine the binding between two proteins. It is very quick to use a cDNA library in order to screen multiple preys at the same time.

The invention also relates to the complexes that are made of a protein coded by one of SEQ ID No 1 to SEQ ID No 150, or SEQ ID No 151, 152 or 153, or SEQ ID NO 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 and one of its binding partners.

The invention is also directed towards the promoters of the genes that lead to the cDNAs represented by one of SEQ ID No 1 to SEQ ID No 150, or SEQ ID No 151, 152 or 153, or SEQ ID No 154 to SEQ ID No 196, or SEQ ID No 197 to SEQ ID NO 210, or SEQ ID No 211 to SEQ ID No 229, or SEQ ID No 230 to SEQ ID No 234, or SEQ ID No 235 to SEQ ID No 245. Indeed, the person skilled in the art can map said genes on the chromosomes, especially by using the data released from the Human Genome Project, and can therefore identify the elements of regulation of transcription. This would lead to the possibility of expressing a foreign protein only in a bone-related environment.

The invention also relates to transgenic animals, except for human beings, in the genome of which has been inserted a nucleic acid sequence according to the invention, especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245, at a locus that is not the natural locus. These animals can have a great utility for the development of osteoporosis or bone-related diseases models.

The person skilled in the art is aware of the ways to prepare transgenic animals, especially by homologous recombination on embryonic stem cells, transfer of said stem cells to embryos, selection of the chimeras that are affected at the reproductive level, growth of said chimeras.

The invention also relates to a transgenic non-human mammal having integrated into its genome a nucleic acid sequence according to the invention, especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID NO 235 to SEQ ID No 245, preferably at a locus that is not the natural locus, operatively linked to regulatory elements, wherein expression of said coding sequences increases the level of the related proteins in said mammal relative to a non-transgenic mammal of the same species, said transgenic mammal exhibiting a difference in bone formation and/or regeneration and/or regulation as compared to a non-transgenic animal.

It is also envisioned that the regulatory elements (promoters, enhancers, introns, similar to those that can be used in mammalian expression vectors) may be tissue-specific, which allows over-expression of the proteins only in a specific type of cells. In particular, the person skilled in the art is aware of the different promoters that can be used for this purpose.

The insertion of the construct in the genome of the transgenic animal of the invention may be performed by methods well known by the artisan in the art, and can be either random or targeted. In a few words, the person skilled in the art will construct a vector containing the sequence to insert within the genome, and a selection marker (for example the gene coding for the protein that gives resistance to neomycine), and may have it enter in the Embryonic Stem (ES) cells of an animal. The cells are then selected with the selection marker, and incorporated into an embryo, for example by microinjection into a blastocyst, that can be harvested by perfusing the uterus of pregnant females. Reimplantation of the embryo and selection of the transformed animals, followed by potential back-crossing allow to obtain such transgenic animal. To obtain a “cleaner” animal, the selection marker gene may be excised by use of a site-specific recombinase, if flanked by the correct sequences.

The invention also relates to a transgenic non-human mammal whose genome comprises a disruption of an endogenous nucleic acid sequence according to the invention, a nucleic acid sequence according to the invention, especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245 wherein said disruption comprises the insertion of a selectable marker sequence, and wherein said disruption results in said non-human mammal exhibiting a difference in bone formation and/or regeneration and/or regulation as compared to a wild-type non-human mammal.

In a preferred embodiment, said disruption is a homozygous disruption.

In a preferred embodiment, said homozygous disruption results in a null mutation of the endogenous gene especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID No 235 to SEQ ID No 245.

In a preferred embodiment, said mammal is a rodent, in the most preferred embodiment, said rodent is a mouse. In this case, the disrupted gene is chosen between SEQ ID No 1 to SEQ ID No 75.

The invention also encompasses an isolated nucleic acid comprising a nucleic acid sequence of the invention, especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID NO 230 to SEQ ID No 234, or SEQ ID No 235 to SEQ ID No 245, knockout construct comprising a selectable marker sequence flanked by DNA sequences homologous to said nucleic acid sequence, wherein when said construct is introduced into a non-human mammal or an ancestor of said non-human mammal at an embryonic stage, said selectable marker sequence disrupts the endogenous gene in the genome of said non-human mammal such that said non-human mammal exhibits a difference in bone formation and/or regeneration and/or regulation as compared to a wild-type non-human mammal.

Said construct is used to obtain the animals that have the disrupted copy of the nucleic acid sequence of the invention, and are generally carried on a vector that is also an object of the invention.

The invention also relates to a mammalian host cell whose genome comprises a disruption of an endogenous nucleic acid sequence of the invention, especially one of SEQ ID No 1 to SEQ ID No 150, or SEQ ID No 151, 152 or 153, or SEQ ID No 154 to SEQ ID No 196, or SEQ ID No 197 to SEQ ID No 210, or SEQ ID No 211 to SEQ ID No 229, or SEQ ID No 230 to SEQ ID No 234, or SEQ ID NO 235 to SEQ ID No 245, wherein said disruption comprises the insertion of a selectable marker sequence. Preferably, said disruption is homozygous and leads to a non-expression of the related functional protein (or expression of a non-functional protein).

It is to be noted that the disruption may be obtained by methods known in the art and may be conditional, i.e. only present in specific types of cells, or induced at some moments of the development. The method to achieve such a goal may be to use site specific recombinases such as Cre (recognizing lox sites) or FLP (recognizing FRT sites) recombinases, under the control of cell-specific promoters. These recombinases (especially Cre) have been shown to be suitable for modifications and their activity may be induced by injection of a substrate (such as an hormone). These modifications are known in the art and may be found, for example in Shibata, et al. (1997, Science 278, 120-3).

Therefore, the transgenic animal or the cell of the invention may not show anymore the selectable marker, which may have been removed upon action of the recombinases, that lead to the disruption of the gene. Nevertheless, in the process of obtaining such disruption, a selectable marker has been inserted within the nucleic acid of the invention, mostly to allow selection of the transformed cells.

U.S. Pat. No. 6,087,555 describes one way of obtaining a knock-out mouse, and the general teaching of this patent is incorporated herein by reference (column 5, line 54 to column 10 line 13). In this patent, it is described an OPG knock-out mouse, but the same method applies to any knock-out mouse. The person skilled in the art will also find information in Hogan et al. (Manipulating the Mouse Embryo: a Laboratory Manual, Cold Spring Harbor Laboratory Press, Cold Spring Harbor, N.Y.; 1986).

The animals and “knock-out” cells of the invention may also be used for identification of pharmacologically interesting compounds. Therefore, the invention also relates to a method of screening compounds that modulate osteoporosis and/or osteogenesis and/or bone regeneration, comprising contacting a compound with the non-human mammal or the knock-out host cell of the invention, and determining the increase or difference in osteogenesis and/or bone regeneration into said non-human mammal or said host cell as compared to the level of osteogenesis and/or bone regeneration of said non-human mammal or said host cell prior to the administration of the compound.

Preferred sequences for performing this transgenic and/or knock-out animals are in particular SEQ ID No 7, 9, 11, 28, 31, 46, 47, 48 and/or 65.

All the above developed aspects of the invention may also be performed, in the context of the invention, with specific nucleic acids that are all related in the same families. In the following, all the references relate to GenBank (www.ncbi.nim.nih.gov).

In particular, it is interesting to develop diagnosis tests that follow expression of the different genes of these families, in order to detect osteoporosis. These diagnosis tests are preferably performed on DNA chips that comprise different probes from the different genes of the family. The person skilled in the art can easily optimize the choice of the better genes that can be integrated on the chip.

Methods for screening compounds that interfere with bone development are also a aspect of the invention, as the compound that modulate expression of the genes of the families are good candidate and warrant to be tested on other models.

One can in particular use the same models as developed by the inventors and described in the examples, or use other models of osteoblastic maturation.

Methods of therapy using all or part of the genes or proteins of the family that are disclosed in the present invention are also part of the invention.

In an embodiment, preferred nucleic acids according to the invention are members of the Wnt-frizzled proteins. This family of proteins is well known by the person skilled in the art, and comprises a large number of members, including transcription factors. The sequence of these proteins may be found on genomic library such as GenBank (www.ncbi.nim.nih.gov). Members of this family have been identified, such as SEQ ID No 5, SEQ ID No 80, SEQ ID No 49, SEQ ID No 124, SEQ ID No 177, SEQ ID No 178.

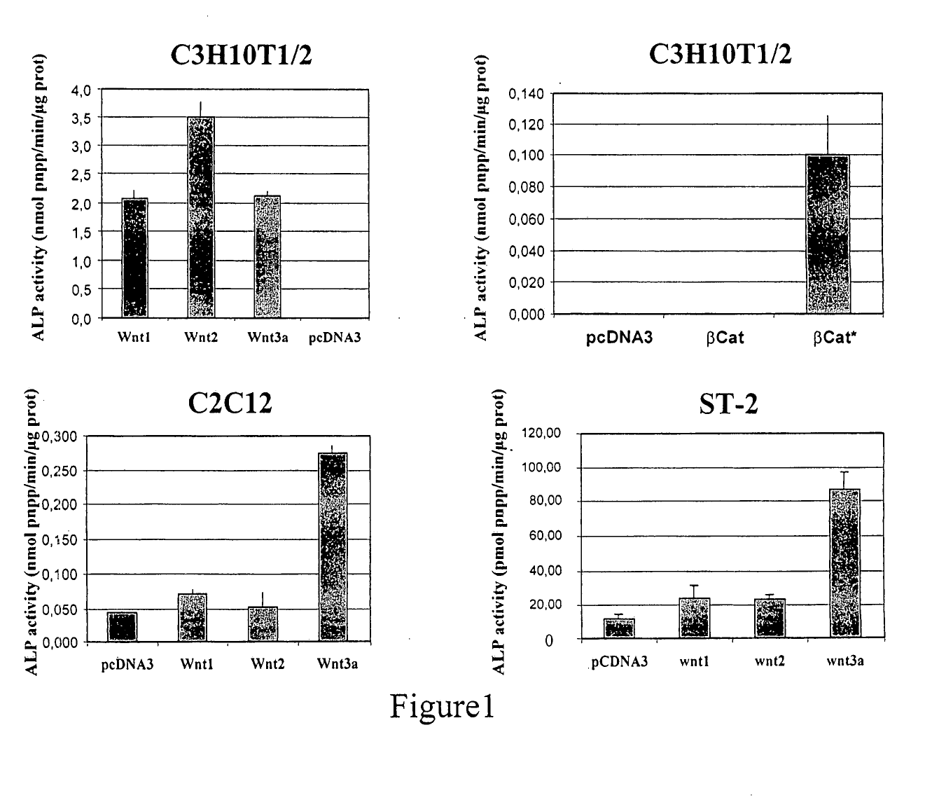

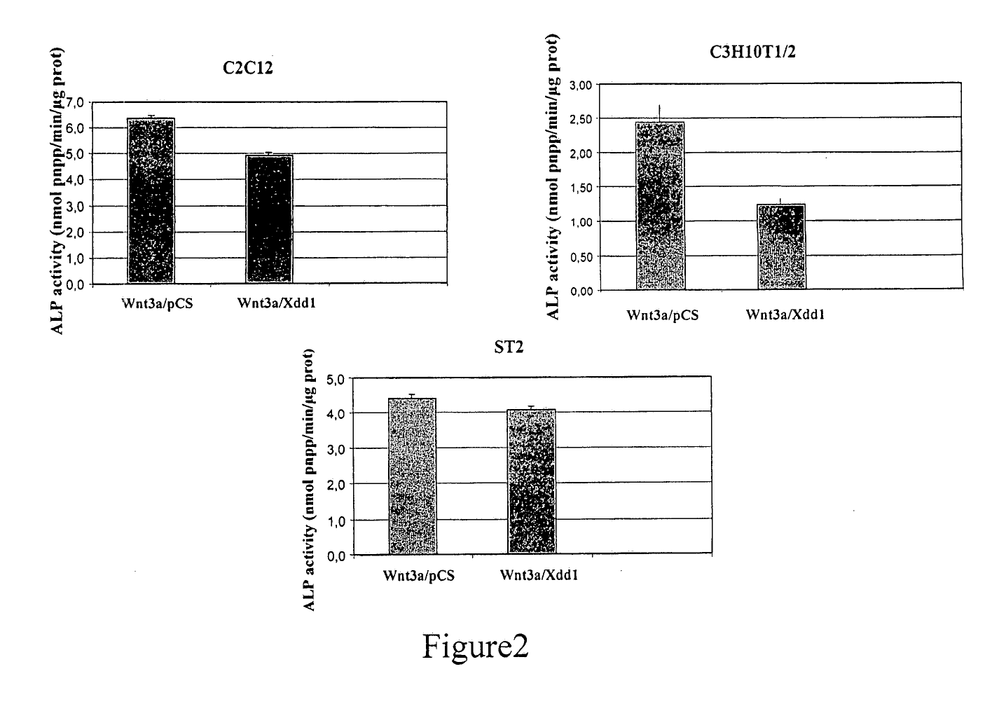

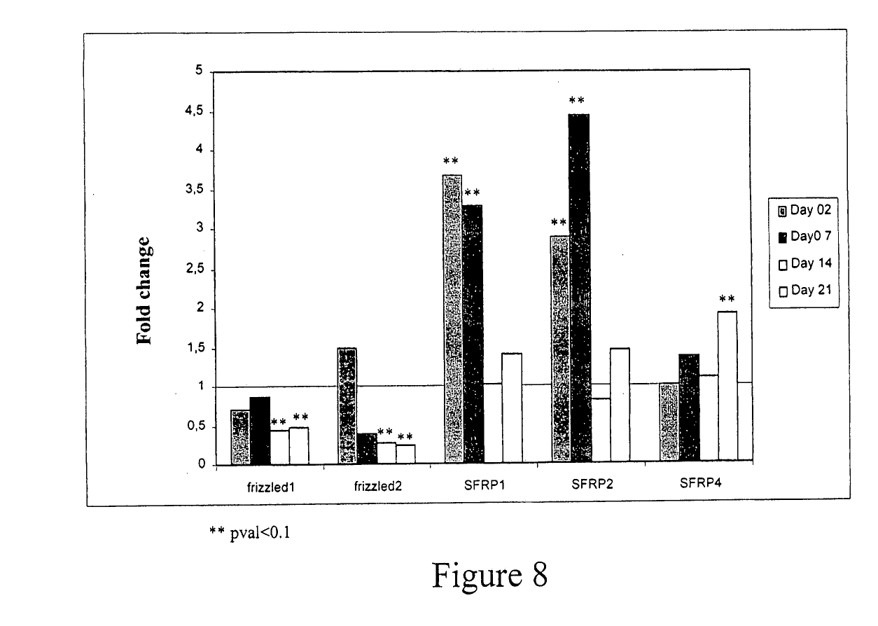

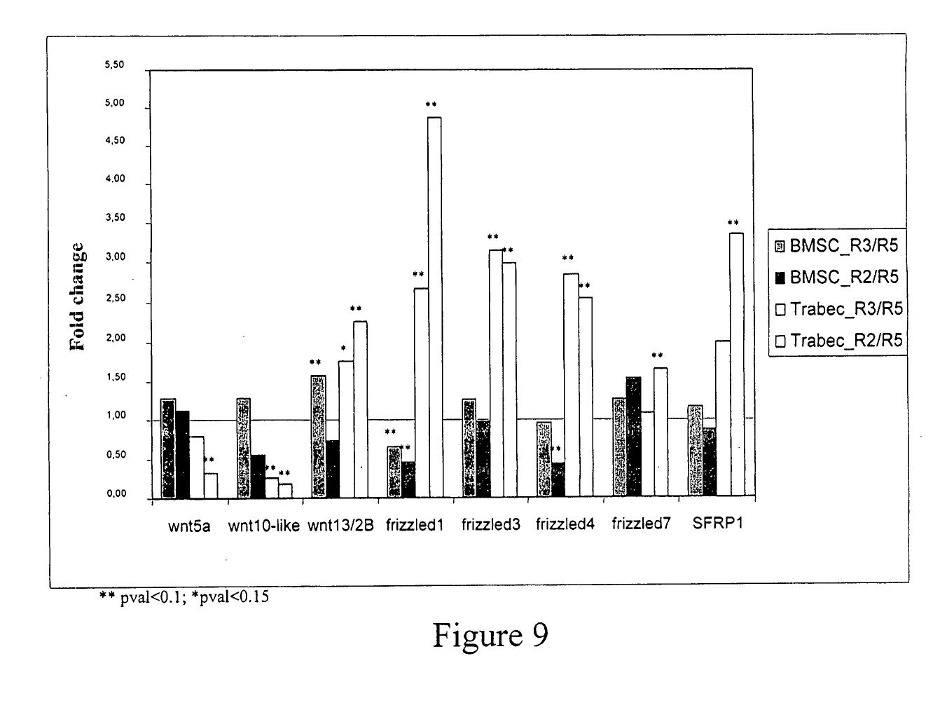

Indeed, the inventors have demonstrated that the members of this family are differentially expressed during bone development, and that there are lots of interactions between expression of members of this family and bone development (FIGS. 1 to 9).

The GenBank sequences of some members of the Wnt-frizzled family are NM_{—}003393 (WNT8B), NM_{—}058244 (WNT8A), transcript variant 2, NM_{—}058238 (WNT7B), NM_{—}004625 (WNT7A), NM_{—}003508 (FZD9), NM_{—}031933 (WNT8A), transcript variant 1, NM_{—}030761 (WNT4), NM_{—}032642 (WNT5B), transcript variant 1, NM_{—}030775 (WNT5B), transcript variant 2, NM_{—}003392 (WNT5A), NM_{—}057168 (WNT16), transcript variant 1, NM_{—}016087 (WNT16), transcript variant 2, NM_{—}003391 (WNT2), NM_{—}033131 (WNT3A), NM_{—}030753 (WNT3), NM_{—}063396 (WNT15), NM_{—}004626 (WNT11), NM_{—}006522 (WNT6), NM_{—}005430 (WNT1), NM_{—}003394 (WNT10B), NM_{—}025216 (WNT10A), NM_{—}003395 (WNT14), NM_{—}024494 (WNT2B), transcript variant WNT-2B2, NM_{—}004185 (WNT2B), transcript variant WNT-2B1, NM_{—}003012 (SFRP1), NM_{—}031866 (FZD8), NM_{—}003014 (SFRP4), NM_{—}017412 (FZD3), NM_{—}012193 (FZD4), NM_{—}007197 (FZD10), NM_{—}001466 (FZD2), NM_{—}003508 (FZD9), NM_{—}003507 (FZD7), NM_{—}003506 (FZD6), NM_{—}003468 (FZD5), NM_{—}003505 (FZD1) NM_{—}003392 (WNT5A), NM_{—}003015 (SFRP5), NM_{—}001463 (FRZB), XM_{—}050625 (SFRP2), NM_{—}031283 (TCF-3), NM_{—}003199 (TCF-4).

In particular, one can cite frizzled 1 (SEQ ID No 205), frizzled 2 (SEQ ID No 206), frizzled 3 (SEQ ID No 207), frizzled 4 (SEQ ID No 208), SFRP1 (SEQ ID No 203), SFRP2 (SEQ ID No 197) and SFRP4 (SEQ ID No 204), wnt1 (SEQ ID No 202), wnt2b (SEQ ID No 198, 199), wnt2 (SEQ ID No 200), wnt3a (SEQ ID No 201), TCF-3 (SEQ ID No 209), TCF-4 (SEQ ID No 210) which are preferred members of the family.

Wnt proteins belong to a large family of cysteine-rich secreted ligands that control development in many organisms from nematodes to mammals. In vertebrates, the Wnt signaling pathway regulates organ development and cellular proliferation, morphology, motility, and cell fate. In the current proposed models, the serine/threonine kinase, GSK-3bl targets cytoplasmic β-catenin for degradation in the absence of Wnt. As a result, cytoplasmic β-catenin levels are low. When Wnt acts on its cell surface receptor Frizzled, dishevelled (Dvl), a cytoplasmic protein, is activated and antagonizes the action of GSK-3. The phosphorylation of β-catenin is reduced and □β-catenin is no longer degraded, resulting in its accumulation in the cytoplasm. Accumulated β-catenin is translocated into the nucleus where it binds to Tcf/Lef, a transcription factor, and stimulates gene expression. In the nucleus, several proteins that bind to Tcf/Lef regulate the complex formation of β-catenin-Tcf-DNA.

Therefore, it appears that β-catenin signaling is regulated in both the cytoplasm and nucleus. Secreted-frizzled related proteins (sFRP) are decoy receptors that are secreted and bind Wnt ligands preventing interactions with frizzled receptors, thus inhibiting Wnt activity. Dkk1, is also a Wnt inhibitor, but unlike sFRP, it does not interact directly with Wnt.

Indeed several Wnt (i.e., Wnt2b and Wnt10a), frizzled (i.e., Fz1, Fz3 and Fz4), sFRP (i.e., sFRP2) and TCF (i.e., TCF1) are regulated in the gene profiling experiments performed by the inventors. Besides, many other players of this same pathway are expressed in cells mentioned above.

The inventors have experimentally showed that overexpression of distinct Wnt, including Wnt1, Wnt2 or Wnt3a increases the production of alkaline phosphatase by pluripotent cells C3H10T1/2, ST2 and C2C12, clearly indicating that those proteins induce osteoblast differentiation (FIG. 1). In addition, overexpression of β-catenin stable mutant, the downstream player of Wnt signaling, also induces osteoblast differentiation of the same cells as determined by measuring the alkaline phosphatase activity.

The involvement of the Wnt pathway in oestoblast differentiation was further evidenced by the fact that Dvl-dominant negative form was able to antagonize the activity of Wnt1, Wnt2 and Wnt3a in pluripotent C3H10T1/2 and C2C12.

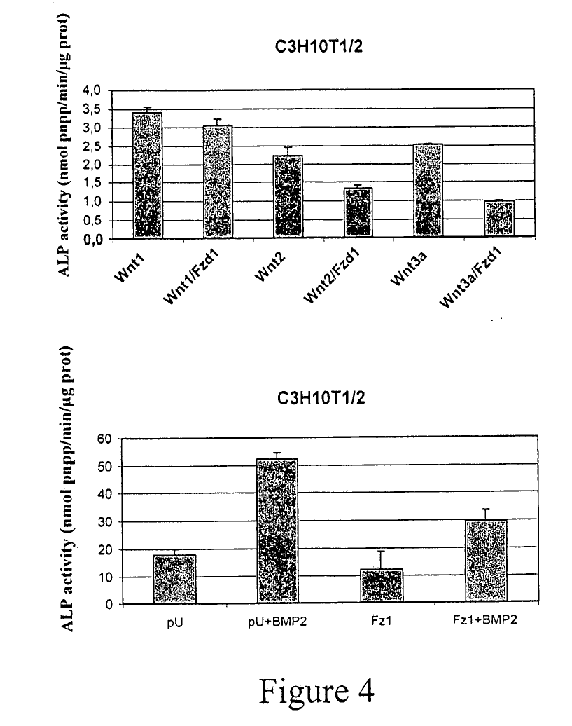

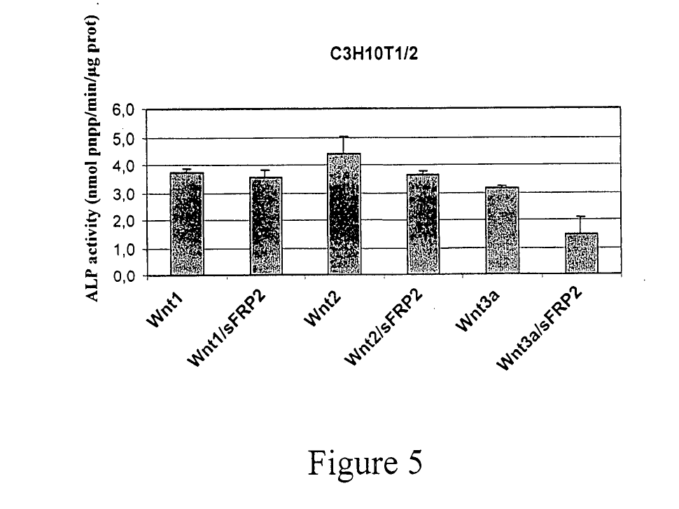

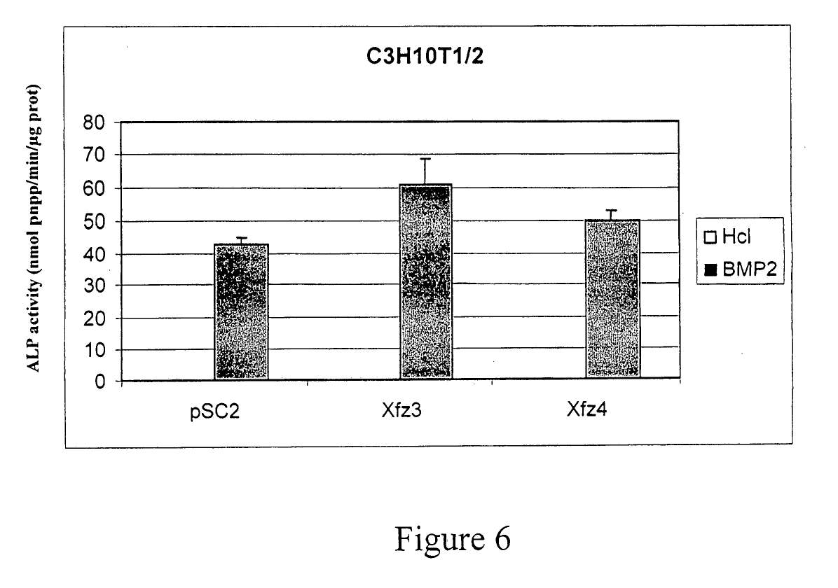

The inventors have also tested the capacity of Fz1 to interact with Wnt proteins and thus affect osteoblast differentiation. Overexpression of Fz1 decreased the activity of Wnt1, Wnt2 and Wnt3a in pluripotent cells C3H10T1/2 as determined by the alkaline phosphatase measurement (FIG. 4). However the expression of Fz3 or Fz4 increased the activity of Wnt proteins in the differentiation process (FIG. 6). In addition, in C3H10T1/2 cells, sFRP2 was able to inhibit Wnt3a-induced alkaline phosphatase, but not Wnt1 and Wnt2 (FIG. 5). This indicate that Frizzled may negatively or positively cooperate with their ligand proteins, Wnt, in these bone-related cells.

We have also showed that Wnt signalling is required for osteoblast differentiation induced by distinct morphogenic proteins including BMP2 and Sonic hedgehog (Shh). In fact, Dvl-dominant negative form inhibited the ability of BMP2 to increase the osteoblast differentiation marker alkaline phosphatase in pluripotent cells C3H10T1/2 and C2C1/2 and the ability of Shh to do the same in C3H10T1/2 cells.

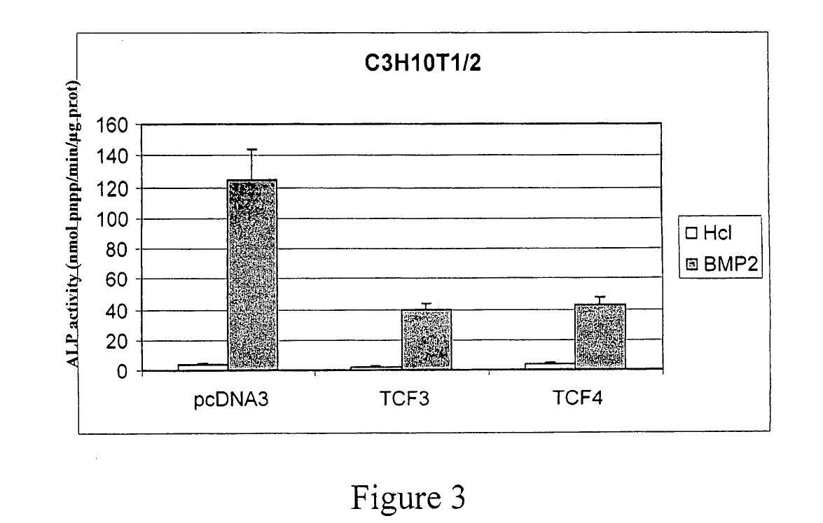

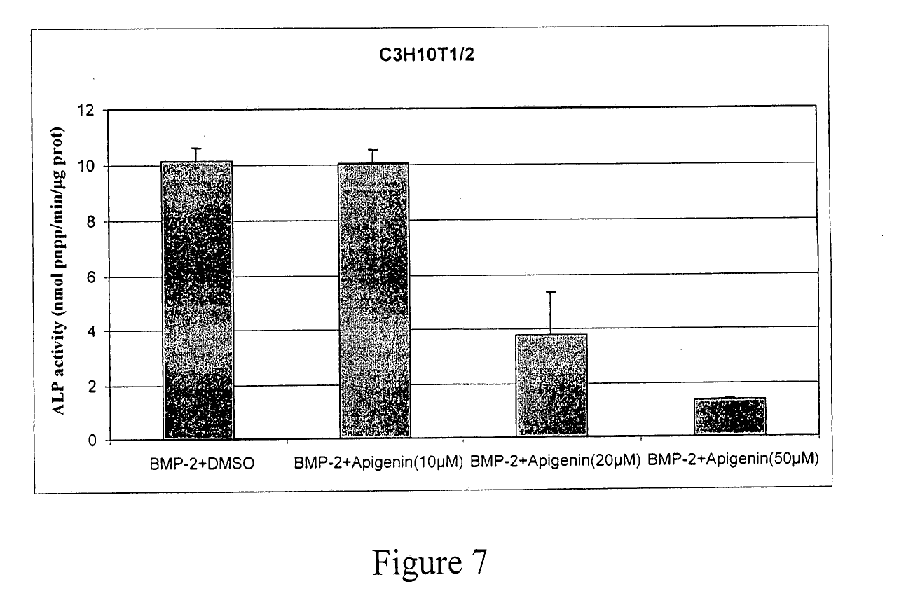

Casein kinase 11, and important kinase in the Wnt signaling pathway, interacts with β-catenin, phosphorylates it and increases its stability and activity. Apigenin, a casein kinase 11 inhibitor, was able to block the activity of BMP-2 and Shh in pluripotent cells C3H10T1/2, ST2 and C2C12 (FIG. 7). The effect of TCF proteins was also investigated, and both TCF3 and TCF4 were found as inhibitors of BMP-2 in C3H10T1/2 cells (FIG. 3).

From this data, it can clearly be stated that Wnt/frizzled is an important pathway that is involved in osteoblast differentiation and bone formation, and that any element within this pathway (extracellular ligands or inhibitors; Frizzled receptors modeling; intracellular signaling; etc) can thus represent a target for drug discovery in the field of osteoporosis, bone remodeling, or any other pathology related to bone formation.

Thus, all the different facets of the invention can be applied to the members of the Wnt-frizzled family as described above, and especially to the particular members that have been pointed out (through the GenBank references, or SEQ ID No 197 to SEQ ID No 210).

It is particularly interesting to use some probes from the Wnt-frizzled family to define a DNA chip, that may be useful for monitoring osteoporosis (diagnosis, evolution of the disease . . . ). It is also interesting to use some members of the family as targets to identify new drugs that interfere with the biological pathway associated with this family, and that can be useful for treating osteoporosis. Methods of screening compounds that are linked to the Wnt-frizzled pathway, and that interfere with the role of Wnt-frizzled proteins during osteogenesis and/or bone loss, especially using read-outs such as the read-outs described above and in the figures (alkaline phosphatase) are particularly interesting.

In this embodiment, one can also cite the proteins that are downstream the Wnt signaling pathway, and in particular the WISP (Wnt1 inducible signaling pathway) proteins. One can cite SEQ ID No 235 and SEQ ID No 236 (WISP1, NM_{—}080838 and NM 003882), SEQ ID No 237 (WISP2; NM_{—}003881)1 SEQ ID No 238 and SEQ ID No 239 (WISP3, NM_{—}130396 and NM_{—}003880).

These proteins are part of the larger CCN family (Perbal, Mol Pathol 2001 April; 54(2):57-79) and are thus known by the person skilled in the art. The family comprises the wisp proteins, as well as CTGF (SEQ ID No 240, NM_{—}001901), CYR61 (SEQ ID No 241, NM_{—}001554), NOV (SEQ ID No 242, NM_{—}002514), and their receptors, that are the multiligand receptor, low density lipoprotein receptor-related protein (LRP), among which one can cite LRP1 (SEQ ID No 243, NM_{—}002332) and LRP2 (SEQ ID No 244, NM_{—}004525). One can also cite LRP3 (SEQ ID No 244, NM_{—}002333) or LRP4 (SEQ ID No 245, XM_{—}035037).

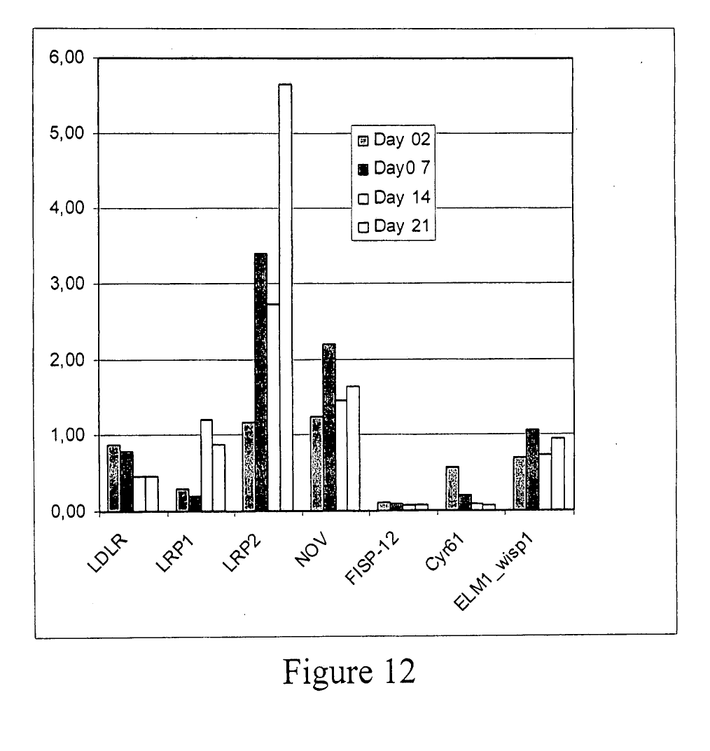

The inventors have demonstrated a regulation of the expression of genes of this family during bone development (osteobalastic maturation) (FIG. 12).

The CCN family of genes encode proteins that participate in fundamental biological processes such as cell proliferation, attachment, migration, differentiation, wound healing, angiogenesis, and several pathologies including fibrosis and tumorigenesis. Whereas CTGF and CYR61 were reported to act as positive regulators of cell growth, NOV (nephroblastoma overexpressed) provided the first example of a CCN protein with negative regulatory properties and the first example of aberrant expression being associated with tumour development. The subsequent discovery of the elm1 (WISP-1), rCOP1 (WISP-2 or CTGF-L), and WISP-3 proteins has broadened the variety of functions attributed to the CCN proteins and has extended previous observations to other biological systems.

Interestingly, WISP CCN-subfamily members WISP1 and WISP2 were identified by using a PCR-based cDNA subtraction strategy performed to discover downstream genes in the WNT signaling pathway. WISP1 and 2 are upregulated in the mouse mammary epithelial cell line transformed by Wnt1, but not by Wnt4.

The multiligand receptor, low density lipoprotein receptor-related protein/alpha(2)-macroglobulin receptor (LRP1). has been recently demonstrated to be a high affinity receptor for CTGF.

The inventors have demonstrated that CTGF and Cyr61 gene expression decreases and NOV expression increases during differentiation of calvaria cells (FIG. 12). CTGF was also found to be down-regulated during the maturation of MC3T3-E1 cells. BMP-2 strongly induces the expression of CTGF in C2C12, C3H10T1/2, ST-2 and MC3T3-E1 cells. In contrast, BMP-2 treatment results in a dramatic decrease of NOV gene expression in C2C12, C3H10T 1/2 and MC3T3-E1 cells. The putative high affinity receptor for CTGF, LRP1 (alpha2 macroglobulin receptor) was found to be down-regulated in the time-course calvaria cultures. LRP1 was also down-regulated in C3H10T1/2, ST-2 and MC3T3-E1 cells treated with BMP-2

Other receptors belonging to the same family that LRP1 were found to be modulated during the process of osteoblast differentiation/maturation. In this way, Whereas LDLR gene expression decreases during maturation of calvaria cells, LRP2 expression was up-regulated in the same primary cells. LDLR expression was also found to be up-regulated in ST-2 and C3H10T1/2 cells treated with BMP-2, specially when cells were co-treated with Shh (a condition reported to enhance the osteoblast commitment of these cells in response to BMP-2). Given the expression and regulation of different members of both CCN and LDL receptors families in osteoblasts and the recent report indicating that LRP1 is the high affinity receptor for CTGF one can speculate that these families might play crucial roles in osteoblast biology.

In another embodiment, preferred nucleic acids according to the invention are part of the Ephin receptors and ephrin family (Eph family).

This family of proteins is well known by the person skilled in the art, and comprises a large number of members, including transcription factors. The sequence of these proteins may be found on genomic library such as GenBank (www.ncbi.nlm.nih.gov). Among some references that relate to this family, one can cite Fox et al., (Oncogene 10 (5), 897-905 (1995)), Lemke (Mol. Cell. Neurosci. 9 (5-6), 331-332 (1997)), Chan et al (Oncogene 6 (6), 1057-1061 (1991)), Bohme et al (Oncogene 8 (10), 2857-2862 (1993)), Boyd et al (J. Biol. Chem. 267 (5), 3262-3267 (1992)).

Preferred members of this family are in particular EphB3, EphB2 and EphA3.

In particular, one can cite SEQ ID No 211 (EphA7, CenBank NM_{—}004440), SEQ ID N⁰ 212 (EphA8, NM_{—}020526), SEQ ID No 213 (EphB4, NM_{—}004444), SEQ ID No 214 (EphB3, NM_{—}004443), SEQ ID No 215 (EphB2, transcript variant 1, NM 004442), SEQ ID No 216 (EphB2, transcript variant 2, NM_{—}017449), SEQ ID No 217 (EphB6, NM_{—}004445), SEQ ID No 218 (EphA4, NM_{—}004438), SEQ ID No 219 (EphA1, NM_{—}005232), SEQ ID No 220 (EphA3, NM_{—}005233), SEQ ID No 221 (ephrin-A4 (EFNA4), NM_{—}005227), SEQ ID No 222 (ephrin-A3 (EFNA3), NM_{—}004952), SEQ ID No 223 (EphA2 (EPHA2), NM_{—}004431), SEQ ID No 224 (ephrin-B2 (EFNB2), NM_{—}004093), SEQ ID No 225 (ephrin-B1 (EFNB1), NM_{—}004429), SEQ ID No 226 (ephrin-A1 (EFNA1), NM_{—}004428), SEQ ID No 227 (ephrin-B3 (EFNB3), NM_{—}001406), SEQ ID No 228 (ephrin-A5 (EFNA5), NM_{—}001962), SEQ ID No 229 (ephrin-A2 (EFNA2), NM_{—}001405).

The Eph receptor proteins (tyrosine kinase receptors) and their ligands, the ephrins, appear to lie functionally at the interface between pattern formation and morphogenesis. As mentioned in GenBank, ephrin receptors and their ligands, the ephrins, mediate numerous developmental processes, particularly in the nervous system. Based on their structures and sequence relationships, ephrins are divided into the ephrin-A (EFNA) class, which are anchored to the membrane by a glycosylphosphatidylinositol linkage, and the ephrin-B (EFNB) class, which are transmembrane proteins. The Eph family of receptors are divided into 2 groups based on the similarity of their extracellular domain sequences and their affinities for binding ephrin-A and ephrin-B ligands. Ephrin receptors make tip the largest subgroup of the receptor tyrosine kinase (RTK) family.

Both Eph receptors and ephrins are dynamically expressed in a wide range of regions of the vertebrate embryo, in the ectoderm, mesoderm and endoderm. Ephrin-mediated clustering of receptors facilitates autophosphorylation in trans (between receptors) of several tyrosine residues including two in the juxtamembrane (JM) region and one in the activation loop of the kinase domain. Upon ligand-stimulated autophosphorylation of the JM tyrosines, the inhibitory conformation of the JM region is destabilized, the JM phosphotyrosines serve as recruitment sites for proteins containing SH2 domains, and the activation loop becomes phosphorylated for full activity.

EphB3 receptor gene expression was found to be up-regulated by BMP-2 treatment in two pluripotent mesenchymal cell lines C2C12 and C31410T1/2 under conditions which promote the osteoblast commitment of these cells. Interestingly the level of expression of both EphB3 receptor and EphA3 could be correlated with the degree of maturation of nomial human trabecular osteoblasts. EphB3 and EphB2 gene expression was significantly down-regulated during differentiation in vitro of murine calvaria cells. Gene expression analysis demonstrated that in murine calvaria, all the ligands and receptors of this family (except EphrA6 receptor and EphrB6 receptor) were found to expressed. These results demonstrate for the first time the expression of ephrins and Eph receptors in bone cells and the regulation of some members of these families in the course of the osteoblast differentiation/maturation.

No report has previously examined the expression of members of the Eph receptor or ephrin families in the osteoblast cell lineage. The modulation of the expression of several members of these families in either mesenchymal cell lines or primary cells suggest the involvement of these proteins in the process of osteoblast differentiation/maturation. Very interestingly, mice homozygous for a null allele of Ryk (a tyrosine kinase implicated in signalling mediated by Eph receptors) have a distinctive craniofacial appearance, shortened limbs and postnatal mortality due to feeding and respiratory complications associated with a complete cleft of the secondary palate.

The results reported above indicate a role of the members of this family during bone formation. It is thus particularly interesting to use some probes from the Eph family to define a DNA chip, that may be useful for monitoring osteoporosis (diagnosis, evolution of the disease . . . ).

It is also interesting to use some members of the family (and especially the receptors) as targets to identify new drugs that interfere with the biological pathway associated with this family, and that can be useful for treating osteoporosis. Methods of screening compounds that are linked to the Eph pathway, and that interfere with the role of Eph proteins during osteogenesis and/or bone loss are particularly interesting, and the compounds thus identified are good candidate for treatment of osteoporosis.

In another embodiment, preferred nucleic acids according to the invention are part of the receptor-tyrosine kinase of the mammalian Tyro 3 family. Members of this family are descried in particular in Lu et al (Nature 398: 723-728, 1999), Lu et al (Science 293: 306-311, 2001).

The receptor protein-tyrosine kinases (PTKs) of the mammalian Tyro 3 family include Tyro 3 (SEQ ID No 233, GenBank NM_{—}006293, also named Rse, Sky, Brt, Tif, Dtk, Etk-2) Ax1 (SEQ ID No 231 and 232, GenBank NM_{—}001699 and NM_{—}021913, also named Ark, Ufo, Tyro 7) and Mer (SEQ ID No 234, GenBank

NM_{—}006343, also named Eyk, Nyk, Tyro 12). These three receptors are widely expressed in adult tissues, but their function is unknown. They share a distinctive structure, with extracellular regions composed of two immunoglobulin-related domains linked to two fibronectin type-III repeats, and cytoplasmic regions that contain an intrinsic PTK domain. Tyro 3, Ax1 and Mer are present in variable amounts in neural, lymphoid, vascular and reproductive tissues, and in primary and tumour cell lines derived from these sources. The kinase activity of each of the receptors is activated by Gas6, a promiscous ligand that exhibits sequence relatedness to a steroid hormone transport protein designated the sex-hormone-binding globulin. Tyro 3 can also bind and be activated by protein S, an anticoagulant in the blood coagulation cascade whose structure is closely related to that of Gas6, although the extent to which protein S functions as a Tyro 3 ligand in vivo is debated.

The sequence of these proteins may be found on genomic library such as GenBank (www.ncbi.nlm.nih.gov).

Gas6, the product of the growth arrest-specific gene 6 (Gas6, SEQ ID No 230, GenBank NM_{—}000820), is a new member of the vitamin K-dependent protein family. Proteins belonging to this family are characterized by post-translational-carboxylation of certain glutamic acid residues by a carboxylase, using vitamin K as cofactor. The carboxyglutainic acid (Gla)-containing module in prothrombin, coagulation factors VII, IX and X, protein C, protein Z, protein S and Gas6 allows these vitamin K-dependent plasma proteins to bind to negatively charged phospholipid membranes.

Apart from a Gla-domain-dependent interaction with phospholipid membranes, Gas6 also binds as a ligand to the receptor tyrosine kinases Ufo, Sky and Mer by its carboxy-terminal globular G domains. It has been implicated in reversible cell growth arrest, survival 1, proliferation and cell adhesion.

Genetics: Ufo, Sky and Mer triple ko mice display multiple major organ defects and develop autoimmunity with symptoms histologically similar to human rheumatoid arthritis, pemphigus vulgaris (autoimmune disease that affects the skin and mucous membranes), and systemic lupus erythematosus. Females are particularly prone to thromboses and recurrent fetal loss. GAS6 ko mice are protected against arterial and venous thrombosis by enhancing the formation of stable platelet macroaggregates. Partial deletion/mutations of Mer causes retinitis pigmentosa and the rat RCS phenotype (retinal degeneration in which the retinal pigment epithelium (RPE) fails to phagocytose shed outer segments, and photoreceptor cells subsequently die).

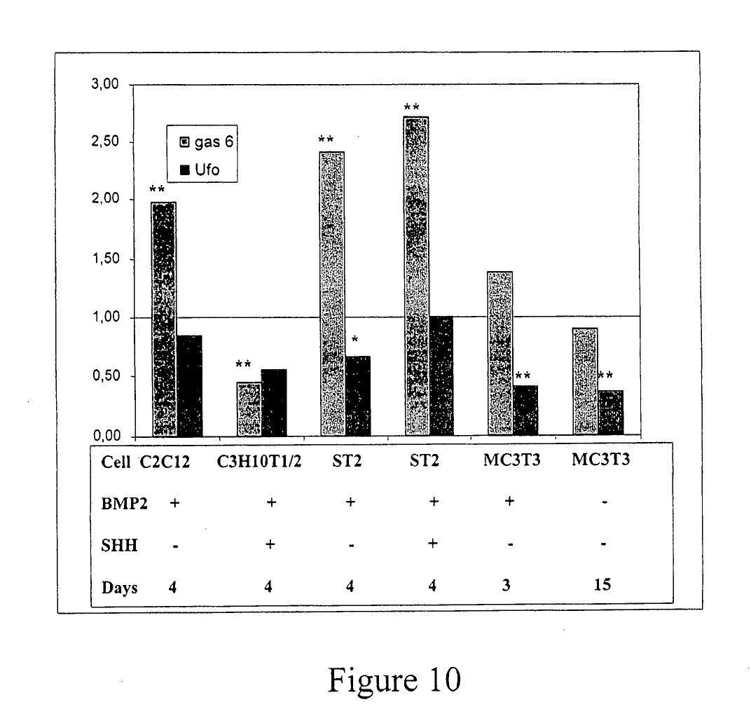

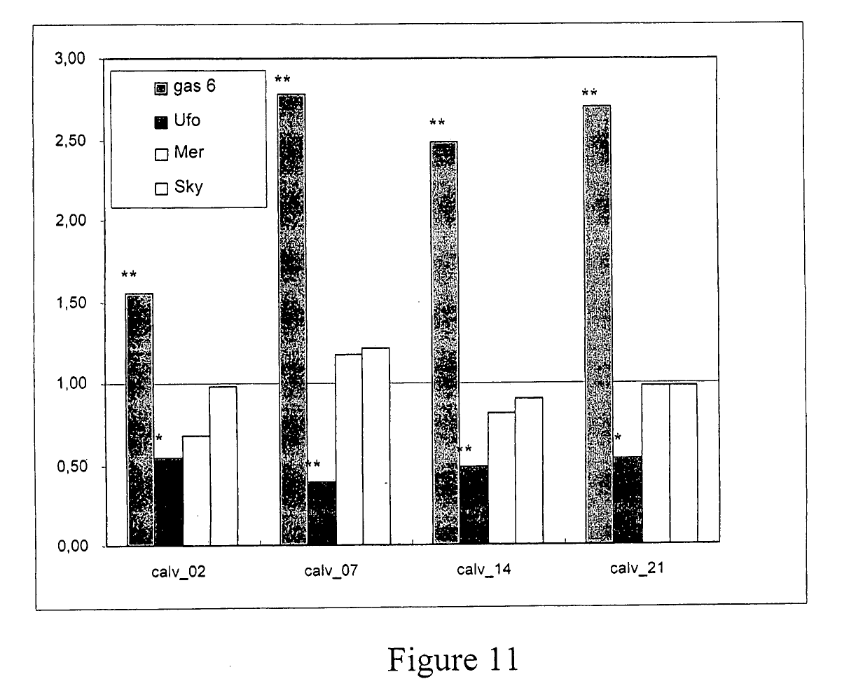

The inventors have observed that Gas6 is significantly upregulated by BMP2 in several of the pre-osteoblast mouse cell lines described in this study (i.e. C2C12, ST2 and MC3T3-E1, FIG. 10) and that gas6 expression is also augmented in maturating primary mouse calvaria cells (FIG. 11) and in maturating NHBC (not shown).

In an opposed way, Ufo/Ax1 has been found to repressed in by BMP2 in C2C12, C3H10T1/2, ST2 and MC3T3-E1 cells as well as during calvaria cells maturation. When overexpressed in C2C12 or C3H10T1/2 cells, Ufo/Ax1 has been found to repress BMP2 induced Alkaline phosphatase activity. Another gas6 receptor, Sky, has been found to be upregulated during NHBC maturation.

The different embodiments of the invention (as described above) may be applied to this family, in particular methods of diagnosis, methods of screening for compounds useful for osteoporosis, using these nucleic acids or proteins as targets or in the test, animal models for osteoporosis and bone formation related diseases, DNA arrays for diagnosis comprising probes originating from these genes, pharmaceutical compositions comprising part or all of the proteins, or antibodies against these proteins, for treating osteoporosis, methods and use of these proteins for treating osteoporosis. . .

The following examples are only as ways of illustration and shall not be considered as restricting the scope of the application.

### EXAMPLES

### Example 1

### Cell Model for Osteopenesis

Mouse cell lines representing different stages of osteoblastic differentiation, C2C12, C2H10T1/2, ST2 and MC3T3-E1, were grown in the presence of agents capable of the induction of osteoblastic differentiation in vitro such as BMP2 and Sonic Hedgehog or in the presence of TGF beta (negative control) or with serum alone. Primary cells derived from mouse calvaria were also cultured for 0, 2, 7 14 and 21 days and RNA prepared for each time point. For example, C3H10T ½ and C2C12 can be obtained from the ATCC (Manassas, Va., USA) under the collection numbers CCL-226 and CRL-1772, respectively.

In the case of testing of a compound for its involvement in osteogenesis, said compound can be added to the cells optionally in addition with the BMP2, and the mRNA can be compared in cells with BMP2 alone, with the compound alone, optionally with BMP2 and the compound, or without any external stimuli.

### Example 2

### Harvesting of mRNA and Preparation of cDNA

Cell extracts for RNA preparation were collected at different time points by using the RNAplus kit provided by Quantum. For every resulting sample, labeled cDNA probe was then generated by reverse transcription followed by in vitro transcription incorporating biotin as part of the standard Affymetrix protocol.

### Example 3

### Determination of the Differential Expression of Genes upon Osteogenesis

The probes were hybridized in duplicate to the complete series of Affymetrix 35K mouse chips (Mu19KsubA, Mu19KsubB, Mu19KsubC, Mu11KsubA, Mu11KsubB and the chips scanned by laser after hybridization and staining. The final data set consisted in of a total of 580 scan files, each obtained using the GeneChip software, which for each qualifier in the file assigns an intensity which is a measure of the corresponding transcript abundance. The output files were further processed into a format which for each intensity adds an estimate of the standard deviation of the noise (Teilhaber et al, 2000, J. Comp. Biol).

A list of possible candidates of genes involved in the regulation of osteoblast differentiation and osteogenesis was established with stringent criteria including the repetition of the observed regulation event in several distinct cell lines, the putative biological relevance of the gene and its expression/regulation in primary mouse calvaria cells. From that list 74 candidates were ultimately selected for full length cloning.

The corresponding human sequences were identified in public data bases with the exception of targets # 13, #60, 61, 62, and # 75, and # 153 that were cloned in-house.

The cloning of the full cDNAs was performed according to the methods described in example 5. Example 4: brief description of isolation of human candidate genes Human candidate genes were identified from 3 distinct experiments derived from primary human cells. The cells were derived from human bone marrow aspirates or from trabecular bone biopsies. Cells were grown using standard protocols and labeled with two distinct antigens STRO1 and Alkaline phosphatase, reflecting different cell stages towards osteoblastic differentiation. Labeled cells were subjected to Facs purification by cell sorting and RNA extracts prepared from the different purified populations by using the RNAplus kit provided by Quantum.

For every resulting sample, labeled cRNA probe was then generated by reverse transcription followed by in vitro transcription incorporating biotin as part of the standard Affymetrix protocol.

The probes were hybridized in duplicate to the complete series of 42K human set of Affymetrix chips (Hu35KA, Hu35 KB, Hu35KC, Hu35 KD, Hu6800. The final data set consisted in of a total of 120 scan files, each obtained using the GeneChip software, which for each qualifier in the file assigns an intensity which is a measure of the corresponding transcript abundance. The output files were further processed into a format which for each intensity adds an estimate of the standard deviation of the noise (Teilhaber et al, 2000, J. Comp. Biol).

A list of possible candidates was established with stringent criteria including the repetition of the same regulation event in several cell populations. From that list 43 candidates (SEQ ID No 154 to SEQ ID No 196) were ultimately selected for full length cloning.

### Example 5

### Brief Description of the Cloning Methods for Full Length DNA

These methods are well known by the person skilled in the art and only their principle will be recalled below.

RT-PCR

The RT-PCR method employs the selective conversion of an mRNA to first strand cDNA through the use of recombinant reverse transcriptase and then subsequent amplification of the cDNA is achieved through a traditional (PCR) polymerase chain reaction using thermostable (Taq) DNA polymerase. The technique is a common molecular biology technique used to amplify specific cDNA sequences from complex mixtures of RNA using gene specific oligonucleotides to prime first strand cDNA synthesis.

Gene Trapper Positive cDNA Selection

The Gene Trapper Positive cDNA Selection (LifeTechnologies) is a method which captures specific cDNA clones through solution hybridization of a biottinylated gene-specific oligonucleotide to a single stranded plasmid DNA preparation and subsequent selection with paramagnetic beads. In this method, an oligonucleotide, complimentary to a defined sequence of the target cDNA is biotinylated at the 3′ end. The biotinylated oligonucleotide is added to a complex mixture of single strand cDNA clones. Specific hybridization between the biotinylated oligonucleotide and the single strand cDNA clone is formed in solution and then captured on streptavidin coated paramagnetic beads. A magnet is used to retrieve the beads from solution with the target cDNA clone attached. The technique is widely used and efficient method to enrich for desired cDNA clones from complex mixtures of library cDNA.

5′ RACE

The 5′ Rapid Amplification of cDNA Ends (RACE) is technique typically employed to clone full length cDNA sequences when only a partial cDNA sequence is initially available. The method typically which employs anchored PCR between a defined sequence within an mRNA and the 5′ end of the mRNA transcript. A unique gene-specific oligonucleotide is used to prime first strand cDNA synthesis from either mRNA source for subsequent PCR amplification. A defined sequence is then added to the 3′ end of the first strand cDNA by tailing with recombinant Terminal Deoxynucleotidyl Transferase (rTdT) or by ligation of an oligonucleotide adapter. Direct amplification of the cDNA between the adapter and gene specific oligonucleotide is achieved through a traditional (PCR) polymerase chain reaction using thermostable (Taq) DNA polymerase.

### Example 6

### Screening of Drugs Modulating the Expression of the Genes in the Invention in Particular in the Model of Example 1

The invention also features a method of screening candidate compounds for the ability to modulate the effective local or systemic concentration or level of a protein according to the invention in an organism.

The method is practiced by

a) incubating one or more candidate compound(s) with cells from a test tissue type of an organism, or in a cell model of osteogenesis, known to produce said given protein for a time sufficient to allow the compound(s) to affect the production, i.e., expression and/or secretion, of said protein by the cells;

b) and then assaying cells and the medium conditioned by the cells for a change in a parameter indicative of the level of production of the protein.

The procedure may be used to identify compounds showing promise as drugs for human use capable of increasing or decreasing production of said protein according to the invention in vivo, thereby to correct or alleviate a diseased condition.

Preferred methods for determining the level of or a change in the level of a protein according to the invention in a cultured cell include using an antibody specific for said protein, e.g., in an immunoassay such as an ELISA or radioimmunoassay; and/or determining the level of nucleic acid, most particularly mRNA, encoding the protein using a nucleic acid probe that hybridizes under stringent conditions with the protein RNA, such as in an RNA dot blot analysis.

Where a change in the presence and/or concentration of the protein of the invention is being determined, it will be necessary to measure and compare the levels of protein in the presence and absence of the candidate compound.

The nucleic acid probe may be a nucleotide sequence encoding the protein or a fragment large enough to hybridize specifically only to RNA encoding a specific protein under stringent conditions, i.e. conditions in which non-specific hybrids will be eluted but at which specific hybrids will be maintained.

The screening method of the invention provides a simple method of determining a change in the level of a protein of the invention, or the level of mRNA production as a result of exposure of cultured cells to one or more compound(s).

The level of said protein in a given cell culture, or a change in that level resulting from exposure to one or more compound(s) indicates that direct application of the compound modulates the level of the protein expressed by the cultured cells. If, for example, a compound upregulated the production of a protein according to the invention, that had been shown as being up-regulated in the osteogenesis cell line model as described in example 1 upon stimulation with BMP2, it would then be desirable to test systemic administration of this compound in an animal model to determine if it upregulated said protein in vivo, and/or promotes osteogenesis in vivo.

If this compound did upregulate the endogenous circulating levels of said protein, it would be consistent with administration of the compound systemically for the purpose of correcting bone metabolism diseases such as osteoporosis, preventing some form of bone degeneration and/or restoring the low density bone to its normal healthy level.

It is important to note that the level of any protein according to the invention in the body may be a result of a wide range of physical conditions, e.g., tissue degeneration, or also as a result of the normal process of aging.

The assay of the invention therefore involves screening candidate compounds for their ability to modulate the effective systemic or local concentration of a protein according to the invention by incubating the compound with a cell culture that has been shown to modulate the level of production of said protein under osteogenesis conditions, and assaying the culture for a parameter indicative of a change in the production level of the protein.

Useful candidate compounds then may be tested for in vivo efficacy in a suitable animal model. These compounds then may be used in vivo to modulate effective protein concentrations in the disease treatment.

The methods for assessing protein production are described in examples 7 and 8.

### Example 7

### Northern Blot

Using specific oligonucleotides probes, transcripts can be identified in mammalian tissues, using standard methodologies well known to those having ordinary skill in the art.

Briefly, total RNA from mouse embryos and organs from post-natal animals is prepared using the acid guanidine thiocyanate-phenolchloroform method (Chomczynski et al., Anal. Biochem. 162:156-159, 1987).

The RNA may be dissolved in TES buffer (10 mM Tris-HCl, 1 mM EDTA, 0.1% SDS, pH 7.5) and treated with Proteinase K (approx. 1.5 mg per g tissue sample) at 45° C. for 1 hr Poly(A) RNA selection on oligo(dT)-cellulose (Type 7, Pharmnacia LKB Biotechnology Inc., Piscataway, N.J.) may be done in a batch procedure by mixing 0.1 g oligo(dT)-cellulose with 11 ml RNA solution (from 1 g tissue) in TES buffer and 0.5M NaCl). Thereafter the oligo(dT) cellulose is washed in binding buffer (0.5M NaCl, 10 mM Tris-HCl, 1 mM EDTA, pH 7.5) and poly(A)⁺ RNA is eluted with water. Poly(A)⁺ RNA (5 or 15 μg/lane) is fractionated on 1 or 1.2% agarose-formaldehyde gels (Selden, in Current Protocols in Molecular Biology, Ausubel et al. eds., pp. 1-4, 8, 9, Greene Publishing and Wiley-Interscience, New York, 1991). 1 μl of 400 μg/ml ethidium bromide is added to each sample prior to heat denaturation (Rosen et al., Focus 12:23-24, 1990). Following electrophoresis, the gels are photographed and the RNA is blotted overnight onto Nytran nitrocellulose membranes (Schleicher & Schuell Inc., Keene, N.M.) with 10×SSC. The membranes are baked at 80° C. for 30-60 min and irradiated with UV light (I mW/cm² for 25 sec). The Northern hybridization conditions may be as previously described (Ozkaynak et al., EMBO J. 9:2085-2093, 1990). For re-use, the filters may be deprobed in 1 mM Tris-HCl, 1 mM EDTA, 0.1% SDS, pH 7.5, at 90-95° C. and exposed to film to assure complete removal of previous hybridization signals.

This leads to a semi-quantitative data, that can be useful to determine the differential expression of the protein of the invention.

### Example 8

### Determination of the Level of a Protein Coded by One of SEQ ID No 1 to SEQ ID No 150, SEQ ID No 151, SEQ ID No 152. SEQ ID No 153 and SEQ ID No 154 to SEQ ID No 196, or SEQ ID No 197 to SEQ ID No 210, or SEQ ID No 211 to SEQ ID No 229 or SEQ ID No No 234, or SEQ ID No 235 to SEQ ID No 245

The level of protein production by the chosen cell type is determined with and without incubating the cell in culture with the compound, in order to assess the effects of the compound on the cell's ability to synthesize or secrete the protein. This can also be accomplished by a direct detection of the level of production of the protein.

Samples for testing the level of protein production include culture supernatants or cell lysates, collected periodically and evaluated for production by immunoblot analysis of a portion of the cell culture itself, collected periodically and used to prepare polyA⁺ RNA for RNA analysis (Sambrook et al., eds., Molecular Cloning, 1989, Cold Spring Harbor Press, Cold Spring Harbor, N.Y.).

To monitor de novo protein synthesis, some cultures are labeled with ³⁵S_methionine/³⁵S-cysteine mixture for 6-24 hours and then evaluated for protein production by conventional immunoprecipitation methods (Sambrook et al., eds., Molecular Cloning, 1989, Cold Spring Harbor Press, Cold Spring Harbor, N.Y.). Alternatively, the production of protein or detemination of the level of protein production may be ascertained using a simple assay for a parameter of cell growth, e.g., cellular proliferation or death. For example, where a protein is produced by a cultured cell line, the addition of antibody specific for said protein may result in relief from protein inhibition of cell growth. Thus, measurement of cellular proliferation can be used as an indication of protein production by a tissue.

In order to quantify the production of a specific protein by a cell type, an immunoassay may be performed to detect said protein using a polyclonal or monoclonal antibody specific for that protein (see Examples 9 and 10).

1 μg/100 μl of affinity-purified polyclonal rabbit IgG specific for the protein of the invention is added to each well of a 96-well plate and incubated at 37° C. for an hour. The wells are washed four times with 0.16M sodium borate buffer with 0.15M NaCl (BSB), pH 8.2, containing 0.1% Tween 20. To minimize non-specific binding, the wells are blocked by filling completely with 1% bovine serum albumin (BSA) in BSB for 1 hour at 37° C. The wells are then washed four times with BSB containing 0.1% Tween 20. A 100 μl aliquot of an appropriate dilution of each of the test samples of cell culture supernatant is added to each well in triplicate and inctibited at 37° C. for 30 min. After incubation, 100′; blotnylated rabbit anti-protein serum (stock solution is about 1 mg/ml and diluted 1:400 in BSB containing 1% BSA before use) is added to each well and incubated at 37° C. for 30 min. The wells are then washed four times with BSB containing 0.1% Tween 20. 100 μl strepavidin-alkaline (Southern Biotechnology Associates, Inc. Birmingham, Ala., diluted 1:2000 in BSB containing 0.1% Tween 20 before use) is added to each well and incubated at 37° C. for 30 min. The plates are washed four times with 0.5M Tris buffered Saline (TBS), pH 7.2. 50 μl substrate (ELISA Amplification System Kit, Life Technologies, Inc., Bethesda, Md.) are added to each well incubated at room temperature for 15 min. Then, 50 μl amplifier (from the same amplification system kit) is added and incubated for another 15 min at room temperature. The reaction is stopped by the addition of 50 μl 0.3M sulphuric acid. The OD at 490 nm of the solution in each well is recorded. To quantitate protein in culture media, a standard curve is performed in parallel with the test samples.

### Example 9

### Preparation of Polyclonal Antibodies

Polyclonal antibody is prepared as follows. Each rabbit is given a primary immunization of 100 μg/500 μl recombinant protein of the invention in 0.1% SDS mixed with 500 μl Complete Freund's Adjuvant. The antigen is injected subcutaneously at multiple sites on the back and flanks of the animal. The rabbit is boosted after a month in the same manner using incomplete Freund's Adjuvant. Test bleeds are taken from the ear vein seven days later.

Two additional boosts and test bleeds are performed at monthly intervals until antibody against the protein of the invention is detected in the serum using an ELISA assay. Then, the rabbit is boosted monthly with 100 μg of antigen and bled (I 5 ml per bleed) at days seven and ten after boosting.

### Example 10

### Preparation of Monoclonal Antibodies

Monoclonal antibody specific for a given protein of the invention may be prepared as follows. A mouse is given two injections of recombinant protein of the invention. The first injection contains 100 μg of said protein in complete Freund's adjuvant and is given subcutaneously. The second injection contains 50 μg of the protein in incomplete adjuvant and is given intraperitoneally. The mouse then receives a total of 230 μg of protein in four intraperitoneal injections at various times over an eight month period. One week prior to fusion, the mouse is boosted intraperitoneally with 100 μg of protein. This boost is repeated five days (IP), four days (IP), three days (IP) and one day (IV) prior to fusion. The mouse spleen cells are then fused to myeloma (e.g., 653) cells at a ratio of 1:1 using PEG 1500 (Boehringer Mannheim), and the cell fusion is plated and screened for specific antibodies using recombinant protein or peptides derived from said protein as antigen. The cell fusion and monoclonal screening are according to procedures widely available in the art. The neutralizing monoclonal is identified by its ability to block the biological activity of the protein when added to a cellular assay which responds biologically to added protein.

### Example 11

### Compounds to Test According to the Invention

The screening methods of the invention is used to test compounds for their effect on the production of morphogenic protein by a given cell type. Examples of compounds which may be screened include but are not limited to chemicals, biological response modifiers (e.g., lymphokines, cytokines, hormones, or vitamins), plant extracts, microbial broths and extracts medium conditioned by eukaryotic cells, body fluids, or tissue extracts.

### Example 12

### Double Hybrid Assay

The double hybrid assay is intended to find the binding partners of a given protein. It may be performed on any protein coded by one of SEQ ID No 1 to SEQ ID No 196 in a system derived from Finley and Brent (Interaction trap cloning with yeast, 169-203, in DNA Cloning, Expression Systems: a practical Approach, 1995, Oxford Universal Press, Oxford), using said protein as a bait and a cDNA library to find the preys.

The protein bait is cloned in plasmid pEG202 known from the person skilled in the art for such a purpose (promoter 67-1511, lexA 1538-2227, ADH Ter 2209-2522, pBR remnants 2540-2889, 2μ ori 2890-4785, YSCNFLP 4923-5729, HIS3 7190-5699, TYIB 7243-7707, RAF_part 7635-7976, pBR backbone 7995-10166, bla 8131-8988).

cDNA of the library are cloned in plasmid pJG4-5, also well known by the person skilled in the art (promoter GAL 1-528, fusion cassette 528-849, ADH Ter 867-1315, 2μ orn 1371-3365, TRP1 3365-4250, pUC backbone 4264-6422, Ap 4412-5274).

Reporting plasmid pSH18-34 is also used. It is in particular available from Invitrogen, under reference number V611-20, et may also already be introduced in strain EGY48 (also called RFY 231), in the same supplier (reference strain alone: C835-00, transformed by pSH18-34: C836-00)

The binding is demonstrated in yeast strain RFY 231 (described in Finley Jr, et al, 98, Proc Natl Acad Sci USA, 95, 14266-71). This yeast strain harbors the following genotype (MATcc Irp1Δ::hisG his3 ura3-1 leu2::3Lexop-LEU2), and is derived from EGY48 (Guris et al., 1993, Cell, 75, 791-803).

The reporting gene is LacZ.

The study is performed on a medium containing galactose, no leucine, and the presence of colored colonies on the plates is studied.

### Example 13

### In Vitro Validation of Some Targets

The candidates genes are cloned in expression vectors and their ability to express a protein of the expected size verified by COS cells transient transfection. The genes are then transfected in C3H10T1/2 cells and/or C2C12 cells (transient over-expression) and positive or negative cooperation with BMP2 is evaluated by the measurement of alkaline phosphatase (enzymatic assay and TaqMan) at 48 hours. Controls of positive cooperation, recombinant Sonic Hedgehog, or negative cooperation, recombinant Noggin, are included in each test.

The following sequences have shown effects in the above described test, at 48th:

| | | Validation in | |
|---|---|---|---|
| SEQ ID No | Name | C2H10T1/2 | Status |
| 7 | SLP1 | + coop over BMP2 | confirmed |
| 9 | P85 | − coop over BMP2 | confirmed |
| 11 | fibromodulin | − coop over BMP2 | confirmed |
| 28 | Meltrin beta | − coop over BMP2 | confirmed |
| 31 | Stomatin/EBP72 | − coop over BMP2 | confirmed |
| 46 | Edg1 | + coop over BMP2 | confirmed |
| 47 | Prostaglandin E | + coop over BMP2 | confirmed |
| | receptor (EP4) | | |
| 48 | Vzg1/Edg2 | + coop over BMP2 | Confirmed |
| 65 | Sprouty | + coop over BMP2 | confirmed |

For targets 46 and 48, a HTS program based on the use of cell lines expressing edg1 or edg2 has been initiated. For Sprouty (target 65) a search for a partner protein is ongoing with a Yeast-2-Hybrids approach.

### Example 14

### In Vitro Validation of Some Targets

Cells were obtained from the calvariae of neonatal mice 1-2 days after birth by sequential collagenase digestion at 37° C. The cells released between 20-40 minutes of collagenase digestion were collected and cultured in proliferation medium (DMEM supplemented with 20% FCS and 2 mM glutamine) until 80% confluence (time 0) and proliferation medium was replaced by differentiation medium (aMEM containing 10% FCS, 2 mM glutamine, 50 microg/ml ascorbic acid and 10 mM beta-glycerolphosphate). Total RNAs were extracted at days 0, 2, 7, 14, and 21 and labeled cRNA probes were generated by reverse transcription followed by in vitro transcription incorporating biotin labeling, according to the standard Affymetrix protocol.

The following sequences have shown effects in the tests describes in Examples 13 and 14:

| | | Validation in vitro | | Regulation in other | |
|---|---|---|---|---|---|
| SEQ | | (coop over BMP2) | | models | |
| ID No | Name | C3H10T1/2 | C2C12 | Calvaria | NHBC/BMSC |
| 2 | TSC-36 (Fstl) | | | down | Up NHBC |
| 5 | SFRP2 | −coop over | | Up/down | |
| | | Wnt3a | | | |
| 6 | PEDF | | | | Up in NHBC |
| 7 | SLP1 | + | | Up | |
| 9 | P85 | + | | down | Up in NHBC |
| 11 | fibromodulin | − | − | down | Up in NHBC |
| 12 | osteomodulin | | | | Down NHBC |
| 16 | Fisp12/CTGF | | | down | Up NHBC & |
| | | | | | BMSC |
| 23 | ADAMTS-1 | | | down | Down NHBC |
| 24 | Cystatin c | − | | Up | Up NHBC |
| | | | | | Down BMSC |
| 26 | BMP1 | − | | down | Up NHBC |
| 27 | Na + K ATPase | | | Up | Up NHBC |
| | Beta3 subunit | | | | |
| 28 | Meltrin beta | − | − | | |
| 30 | Metalloproteinase | − | − | | Up NHBC |
| | 14 | | | | |
| 31 | Stomatin/EBP72 | − | | | Down NHBC |
| 32 | NOV | | | up | Down NHBC |
| 34 | biglycan | | | down | Up NHBC |
| 35 | Fibulin-4 | | | down | Up NHBC |
| 36 | Annexin II | | | down | Up NHBC |
| 37 | Tyr kinase UFO | − | − | down | |
| 40 | Bone marrow | | | up | Up in NHBC |
| | stromal antigen2 | | | | |
| 41 | Macrophage | | | down | Up in NHBC |
| | mannose receptor | | | | |
| | typec | | | | |
| 42 | Mac2 antigen/ | − | − | up | Up NHBC |
| | galectin 3 | | | | Down BMSC |
| 43 | KIAA0620 | | | up | Up NHBC |
| 45 | Taurine/beta- | − | | up | |
| | alanine transporter | | | | |
| 46 | Edg1 | + | | | |
| 47 | Prostaglandin E | + | | | |
| | receptor (EP4) | | | | |
| 48 | Vag1/Edg2 | + | | | Up NHBC |
| 49 | Frizzled 1 | − | − | | Up NHBC |
| 51 | Pkd2 | | | down | Down NHBC |
| 53 | AEBP1 | | | down | Up NHBC |
| | | | | | Down BMSC |
| 54 | Mevalonate kinase | | | | Up NHBC & |
| | | | | | BMSC |
| 55 | MSP23/Osf3 | − | − | up | |
| 56 | FKBP65/63 | − | | down | Down BMSC |
| 57 | Nedd4-like | | | down | Up NHBC |
| 58 | TSC-22 | | | | Up NHBC & |
| | | | | | BMSC |
| 63 | Similar to gene 33 | − | − | down | |
| 64 | HMR/NUR77 | | | | Up NHBC |
| 65 | Sprouty | + | | | |
| 67 | Similar alpha- | − | | | Down |
| | actinin-2- | | | | NHBC & |
| | associated | | | | BMSC |
| 68 | SOCS3 | − | − | down | |

For target 65 (Sprouty) the positive cooperation observed over BMP2 was confirmed by an anti-sense approach, i.e. in the presence of the anti-sense oligo the positive cooperation was abolished.

1. A method for diagnosing osteoporosis in a patient, wherein said method comprises:
a) obtaining a biological sample from said patient, and
b) analyzing the gene expression of at least one of SEQ ID NOS. 1-150, SEQ ID NOS. 151, 1522 or 153, SEQ ID NOS. 154-196, SEQ ID NOS. 197-210, SEQ ID NOS. 211-229, SEQ ID NOS. 230-234, or SEQ ID NOS. 235-245 from said sample,
c) detecting optionally-amplified cDNA contained in said sample to diagnose osteoporosis in said patient.

2. The method of claim 1, wherein said gene expression analysis comprises:
a) making complementary DNA (cDNA) from messenger RNA (mRNA) contained in said sample,
b) optionally amplifying portions of said cDNA corresponding to at least one of SEQ ID NOS. 1-150, SEQ ID NOS. 151, 1522 or 153, SEQ ID NOS. 154-196, SEQ ID NOS. 197-210, SEQ ID NOS. 211-229 SEQ ID NOS. 230-234, or SEQ ID NOS. 235-245, and
c) detecting said optionally amplified cDNA, to diagnose osteoporosis.

3. The method of claim 1, wherein said gene expression analysis is performed by using a DNA chip.

4. The method of claim 1, wherein said sample is from a tissue from said patient.

5. The method of claim 1, wherein a labeled specific oligonucleotide primer or probe is used to detect of said cDNA.

6. The method of claim 1, wherein said amplified cDNA is size-separated by electrophoresis prior to detection.

7. The method of claim 6, wherein blotting and autoradiography are performed on said size-separated cDNA.

8. The method of claim 1, wherein said gene expression analysis is performed by analyzing mRNA obtained from cells of said sample.

9. A method for diagnosing osteoporosis in a mammal comprising:
a) contacting a sample of mammalian bone or cartilaginous tissue with an agent for specifically detecting endogenous expression of one of SEQ ID NOS. 1-150, SEQ ID NOS. 151, 152, or 153, SEQ ID NOS. 154-196, SEQ ID NOS. 197-210, SEQ ID NOS. 211-229, SEQ ID NOS. 230-234, or SEQ ID NOS. 235-245 in said tissue sample;
b) detecting a level of endogenous expression of said gene in said tissue; and
c) comparing said level of endogenously expressed gene represented by one of SEQ ID NOS. 1-150, SEQ ID NOS. 151, 1522 or 153, SEQ ID NOS. 154-196, SEQ ID NOS. 197-210, SEQ ID NOS. 211-229, SEQ ID NOS. 230-234, SEQ ID NOS. 235-245 in said tissue with a reference level of said gene represented by one of SEQ ID NOS. 1-150, SEQ ID NOS. 151, 152, or 153, SEQ ID NOS. 154-196, SEQ ID NOS. 197-210, SEQ ID NOS. 211-229, SEQ ID NOS. 230-234, or SEQ ID NOS. 235-245, endogenously expressed in undiseased mammalian bone or cartilaginous tissue to diagnose osteoporosis in said mammal.

10. The method of claim 9, wherein said agent is a nucleic acid probe that hybridizes specifically with RNA transcribed from said gene chosen from SEQ ID NOS. 1-150, SEQ ID NOS. 151, 1524 or 153, SEQ ID NOS. 154-196, SEQ ID NOS. 197-210, SEQ ID NOS. 211-229, SEQ ID NOS. 230-234, or SEQ ID NOS. 235-245, present in cells of said tissue, or with cDNA obtainable from said RNA.

11. The method of claim 9, wherein said agent is a monoclonal or polyclonal antibody that specifically recognizes the protein encoded by said gene chosen from SEQ ID NOS. 1-150, SEQ ID NOS. 151, 152, or 153, SEQ ID NOS. 154-196, SEQ ID NOS. 197-210, SEQ ID NOS. 211-229, SEQ ID NOS. 230-234, or SEQ ID NOS. 235-245.

12. The method of claim 9, further comprising:
d) contacting a sample of said mammalian bone or cartilaginous tissue with a control nucleic acid probe that hybridizes specifically with RNA transcribed from a gene expressed uniformly in mammalian tissues;
e) detecting a level of expression of said gene in said tissue; and
f) comparing the relative gene expression levels of said gene represented by one of SEQ ID NOS. 1-150, SEQ ID NOS. 151, 152, or 153, SEQ ID NOS. 154-2196, SEQ ID NOS. 197-210, SEQ ID NOS. 211-229, SEQ ID NOS. 230-234, or SEQ ID NOS. 235-245 and said gene in said tissue, with the relative gene expression levels of said gene represented by one of SEQ ID NOS. 150, or SEQ ID NOS. 151, 152, or 153, SEQ ID NOS. 154-196, SEQ ID NOS. 197-210, SEQ ID NOS. 211-229, SEQ ID NOS. 230-234, or SEQ ID NOS. 235-2452 and said gene in undamaged or undiseased mammalian bone or cartilaginous tissue.

13. A method for promoting osteogenesis and/or preventing osteoporosis comprising administering to a subject in need thereof a therapeutically effective amount of a protein product encoded by one of SEQ ID NOS. 1-150, SEQ ID NOS. 151, 1522 or 153, or SEQ ID NOS. 154-196, SEQ ID NOS. 197-210, SEQ ID NOS. 211-229, SEQ ID NOS. 230-234, or SEQ ID NOS. 235-245, wherein said protein product promotes osteogenesis and/or prevents osteoporosis.

14. A method for promoting osteogenesis and/or preventing osteoporosis comprising administering to a subject in need thereof a therapeutically effective amount of a nucleic acid comprising one of SEQ ID NOS. 1-150, SEQ ID NOS. 151, 1524 or 153, SEQ ID NOS. 154-196, SEQ ID NOS. 197-210, SEQ ID NOS. 211-229, SEQ ID J NOS. 230-234, or SEQ ID NOS. 235 te SEQ ID N⁰-245, wherein said nucleic acid product promotes osteogenesis and/or prevents osteoporosis.

15. The method of claim 14, wherein said nucleic acid is administered to said subject and wherein said nucleic acid enters said subject's osteoblastic or osteoclastic cells.

16. The method of claim 15, wherein said nucleic acid is introduced into said cells by means of a viral vector.

17. The method of claim 15, wherein said nucleic acid is introduced into said cells by means of a synthetic vector.

18. A method for promoting osteogenesis and/or preventing osteoporosis comprising administering to a subject in need thereof a therapeutically effective amount of an inhibitor of a protein product encoded by one of SEQ ID 1 NOS. 1-150, SEQ ID NOS. 151, 1524 or 153, SEQ ID NOS. 154-196, SEQ ID NOS. 197-210, SEQ ID NOS. 211-229, SEQ ID NOS. 230-234, or SEQ ID NOS. 235-245.

19. The method of claim 18, wherein said inhibitor is a monoclonal or polyclonal antibody directed towards said protein product encoded by one of SEQ ID NOS. 1-105.

20. The method of claim 19, wherein said inhibitor is a nucleic acid, and wherein said inhibitor is antisense to the nucleic acid represented by one of SEQ ID NOS. 1-150.

21. A method for identifying a compound having a role in osteogenesis, comprising the steps of:
a) contacting said compound with a cell model of osteogenesis, and
b) comparing the level of gene expression of one of SEQ ID NOS. 1-150, SEQ ID NOS. 151, 1522 or 153, SEQ ID NOS. 154-196, SEQ ID NOS. 197-210, SEQ ID NOS. 211-229, SEQ ID NOS. 230-234, or SEQ ID NOS. 235-245 in said cell model with the level of gene expression of said gene in said cell model to which said compound has not been brought in contact, wherein the role of said compound in said osteogenesis being is deduced from the difference between said levels of gene expression.

22. A method for identifying a compound useful for modulation of osteogenesis, comprising:
a) contacting said compound with a protein encoded by one of SEQ ID NOS. 1-150, SEQ ID NOS. 151, 152, or 153, SEQ ID NOS. 154-196, SEQ ID NOS. 197-210, SEQ ID NOS. 211-229, SEQ ID NOS. 230 to SEQ ID N2-234, or SEQ ID NOS. 235-245, and
b) analyzing the interaction between said compound and said protein, wherein the utility of said compound in the modulation of osteogenesis is deduced from the presence of the interaction between said compound and said protein encoded by one of SEQ ID NOS. 1-150.

23. A method for identifying a compound useful for treatment of osteoporosis, comprising:
a) performing the method of claim 21,
b) modifying the compound selected in step a),
c) testing the modified compound of step b) in in vitro and/or in vivo models relevant for assessment of osteoporosis,
d) identifying the compound having an anti-osteoporosis activity superior to the compound selected in step a).

24. A method for identifying a compound useful for treatment of osteoporosis, comprising:
a) performing the method of claim 21,
b) modifying the compound selected in step a),
c) testing the modified compound of step b) in in vitro and/or in vivo models relevant for assessment of osteoporosis,
d) identifying the compound having the searched biological effect on osteoporosis, with a reduced toxicity in an animal model than the compound selected in step a).

25. A compound identified by the method of claim 21.

26. An isolated nucleic acid sequence, wherein said nucleic acid sequence is upregulated during osteogenesis, and wherein said nucleic acid sequence is chosen from the group consisting of:
a) one of SEQ ID NOS. 1-9, SEQ ID NOS. 11-20, SEQ ID NO. 27, SEQ ID NOS. 33-36, SEQ ID NOS. 45-50, SEQ ID NO. 53, SEQ ID NO. 54, SEQ ID NOS. 58-62, SEQ ID NO. 66, SEQ ID NOS. 69-75, SEQ ID NOS. 76-84, SEQ ID NOS. 86-95, SEQ ID NO. 102, SEQ ID NOS. 108-111, SEQ ID NOS. 120-125, SEQ ID NO. 128, SEQ ID NO. 129, SEQ ID NOS. 133-137, SEQ ID NO. 141, SEQ ID NOS. 144-150, SEQ ID NO. 156, SEQ ID NOS. 158-161, SEQ ID NOS. 164-167, SEQ ID NOS. 170-174, SEQ ID NO. 176, SEQ ID NO. 177, SEQ ID NO. 178, SEQ ID NOS. 180-185, SEQ ID NO. 187, SEQ ID NOS. 191-194, or SEQ ID NO. 196
b) an isolated and purified nucleic acid comprising the nucleic acid sequence of a)
c) an isolated nucleic acid that specifically hybridizes under stringent conditions to the complement of the nucleic acid sequence of a), wherein said nucleic acid sequence encodes a protein that is upregulated during osteogenesis
d) an isolated nucleic acid having at least 80% homology with the nucleic acid sequence of a), wherein said nucleic acid sequence encodes a protein that is upregulated during osteogenesis
e) a fragment of said nucleic acid sequence of a) comprising at least 15 nucleotides.

27. An isolated nucleic acid sequences wherein said nucleic acid sequence is downregulated during osteogenesis, and wherein said nucleic acid sequence is chosen from the group consisting of:
a) one of SEQ ID NO. 10, SEQ ID NOS. 211-726, SEQ ID NOS. 28-32, SEQ ID NOS. 37-44, SEQ ID NO. 51, SEQ ID NO. 52, SEQ ID NOS. 55-57, SEQ ID NOS. 63-65, SEQ ID NO. 67, SEQ ID NO. 68, SEQ ID NO. 85, SEQ ID NOS. 96-101, SEQ ID NOS. 103-107, SEQ ID NOS. 112-119, SEQ ID NO. 126, SEQ ID NO. 127, SEQ ID NOS. 130-132; SEQ ID NOS. 138-140, SEQ ID NO. 142, SEQ ID NO. 143, SEQ ID NO. 154, SEQ ID NO. 155, SEQ ID NO. 157, SEQ ID NO. 162, SEQ ID NO. 163, SEQ ID NO. 168, SEQ ID NO. 196, SEQ ID NO. 175, SEQ ID NO. 176, SEQ ID NO. 179, SEQ ID NO. 186, SEQ ID NO. 188, SEQ ID NO. 189, SEQ ID NO. 190, or SEQ ID NO. 195
b) an isolated and purified nucleic acid comprising the nucleic acid sequence of a)
c) an isolated nucleic acid that specifically hybridizes under stringent conditions to the complement of the nucleic acid sequence of a), wherein said nucleic acid sequence encodes a protein that is upregulated during osteogenesis
d) an isolated nucleic acid having at least 80% homology with the nucleic acid sequence of a), wherein said nucleic acid sequence encodes a protein that is upregulated during osteogenesis
e) a fragment of the nucleic acid sequence of a) comprising at least 15 nucleotides.

28. An isolated protein or peptide encoded by the nucleic acid sequence of claim 26.

29. A monoclonal or polyclonal antibody that specifically recognizes the protein or peptide of claim 28.

30. A pharmaceutical composition comprising the compound of claim 25 and a pharmaceutically acceptable excipient.

31. A method for the therapy of a bone disease, comprising administering to a subject in need thereof the compound of claim 25.

32. A DNA chip that harbors at least one probe that hybridizes to one of SEQ ID NOS. 1-150.

33. A transgenic non-human mammal having integrated into its genome the nucleic acid sequence of claim 26, operatively linked to regulatory elements, wherein expression of said coding sequence increases the level of said nucleic acid sequence's related protein, and wherein said non-human mammal exhibits a difference in bone formation and/or regeneration compared to a non-transgenic mammal of the same species.

34. A transgenic non-human mammal whose genome comprises a disruption of the nucleic acid of claim 26, wherein said disruption comprises the insertion of a selectable marker sequence, and wherein said disruption results in said non-human mammal exhibiting a difference in bone formation and/or regeneration and/or regulation compared to a non-transgenic mammal of the same species.

35. The transgenic mammal of claim 34, wherein said disruption is a homozygous disruption.

36. The transgenic mammal of claim 35, wherein said homozygous disruption results in a null mutation of the nucleic acid sequence.

37. The mammal of claim 33 wherein said mammal is a mouse.

38. The method of claim 4, wherein said tissue is from a bone, cartilaginous tissue, blood, or bodily fluid.

39. The method of claim 5, wherein said probe is contained on the surface of a DNA chip.

40. The method of claim 39, wherein said DNA chip contains sequences to which said cDNA may hybridize under stringent conditions.

41. A method for identifying a compound useful for treatment of osteoporosis, comprising:
a) performing the method of claim 22,
b) modifying the compound selected in step a),
c) testing the modified compound of step b) in in vitro and/or in vivo models relevant for assessment of osteoporosis,
d) identifying the compound having an anti-osteoporosis activity superior to the compound selected in step a).

42. A method for identifying a compound useful for treatment of osteoporosis, comprising:
a) performing the method of claim 22,
b) modifying the compound selected in step a),
c) testing the modified compound of step b) in in vitro and/or in vivo models relevant for assessment of osteoporosis,
d) identifying the compound having the searched biological effect on osteoporosis, with a reduced toxicity in an animal model than the compound selected in step a).

43. A compound identified by the method of claim 22.

44. A compound identified by the method of claim 23.

45. A compound identified by the method of claim 24.

46. A compound identified by the method of claim 41.

47. A compound identified by the method of claim 42.

48. An isolated protein or peptide encoded by the nucleic acid sequence of claim 27.

49. A monoclonal or polyclonal antibody that specifically recognizes the protein or peptide of claim 48.

50. A pharmaceutical composition comprising the nucleic acid of claim 26 and a pharmaceutically acceptable excipient.

51. A pharmaceutical composition comprising the nucleic acid of claim 27 and a pharmaceutically acceptable excipient.

52. A pharmaceutical composition comprising the protein of claim 28 and a pharmaceutically acceptable excipient.

53. A pharmaceutical composition comprising the antibody of claim 29 and a pharmaceutically acceptable excipient.

54. A method for the therapy of a bone disease, comprising administering to a subject in need thereof the nucleic acid of claim 26.

55. A method for the therapy of a bone disease, comprising administering to a subject in need thereof the nucleic acid of claim 27.

56. A method for the therapy of a bone disease, comprising administering to a subject in need thereof the protein of claim 28.

57. A method for the therapy of a bone disease, comprising administering to a subject in need thereof the antibody of claim 29.

58. A method for the therapy of a bone disease, comprising administering to a subject in need thereof the pharmaceutical composition of claim 30.

59. A transgenic non-human mammal having integrated into its genome the nucleic acid sequence of claim 27, operatively linked to regulatory elements, wherein expression of said coding sequence increases the level of said nucleic acid sequence's related protein, and wherein said non-human mammal exhibits a difference in bone formation and/or regeneration compared to a non-transgenic mammal of the same species.

60. A transgenic non-human mammal whose genome comprises a disruption of the nucleic acid of claim 27, wherein said disruption comprises the insertion of a selectable marker sequence, and wherein said disruption results in said nonhuman mammal exhibiting a difference in bone formation and/or regeneration and/or regulation compared to a non-transgenic mammal of the same species.

61. The transgenic mammal of claim 60, wherein said disruption is a homozygous disruption.

62. The transgenic mammal of claim 61, wherein said homozygous disruption results in a null mutation of the nucleic acid sequence.

63. The transgenic mammal of claim 36, wherein said nucleic acid sequence comprises the nucleic acid sequence of claim 26.

64. The transgenic mammal of claim 36, wherein said nucleic acid sequence comprises the nucleic acid sequence of claim 27.

65. The transgenic mammal of claim 62, wherein said nucleic acid sequence comprises the nucleic acid sequence of claim 26.

66. The transgenic mammal of claim 62, wherein said nucleic acid sequence comprises the nucleic acid sequence of claim 27.

67. The mammal of claim 34, wherein said mammal is a mouse.

68. The mammal of claim 59, wherein said mammal is a mouse.

69. The mammal of claim 60, wherein said mammal is a mouse.
